(19) United States
(12) Patent Application Publication
(10) Pub. No.: US 2014/0128667 A1
(43) Pub. Date: May 8, 2014

(54) ADAPTIVE EMBRYO SELECTION CRITERIA OPTIMIZED THROUGH ITERATIVE CUSTOMIZATION AND COLLABORATION

(71) Applicants: Niels B Ramsing, Risskov (DK); Jens K Gundersen, Viby J (DK); Karen Marie Hilligsøe, Aarhus (DK); Jørgen Berntsen, Viborg (DK); Inge Errebo Agerholm, Brædstrup (DK)

(72) Inventors: Niels B Ramsing, Risskov (DK); Jens K Gundersen, Viby J (DK); Karen Marie Hilligsøe, Aarhus (DK); Jørgen Berntsen, Viborg (DK); Inge Errebo Agerholm, Brædstrup (DK)

(73) Assignee: UNISENSE FERTILITECH A/S, Aarhus N (DK)

(21) Appl. No.: 14/128,295

(22) PCT Filed: Jun. 29, 2012
(86) PCT No.: PCT/DK2012/050236

§ 371 Date: Dec. 20, 2013

(30) Foreign Application Priority Data
Jul. 2, 2011 (DK) PA 2011 70355

(51) Int. Cl. G06F 19/00; A61B 17/435

(52) U.S. Cl. CPC G06F 19/34; A61B 17/435
USPC 600/34; 702/19

(57) ABSTRACT

The present invention relates to a system and a method for determining quality criteria in order to select the most viable embryos after in vitro fertilization. The present invention may further be applied for iteratively adapting embryo quality criteria based on new knowledge, historical selection & fertilization data and cooperation between fertility clinics.

The present invention relates to a system and a method for determining quality criteria in order to select the most viable embryos after in vitro fertilization. The present invention may further be applied for iteratively adapting embryo quality criteria based on new knowledge, historical selection & fertilization data and cooperation between fertility clinics.

### BACKGROUND OF INVENTION

Infertility affects more than 80 million people worldwide. It is estimated that 10% of all couples experience primary or secondary infertility (Vayena et al. 2001). In vitro fertilization (IVF) is an elective medical treatment that may provide a couple who has been otherwise unable to conceive a chance to establish a pregnancy. It is a process in which eggs (oocytes) are taken from a woman's ovaries and then fertilized with sperm in the laboratory. The embryos created in this process are then placed into the uterus for potential implantation. To avoid multiple pregnancies and multiple births only a few embryos are transferred (normally less than four and ideally only one (Bhattacharya et al. 2004)). Selecting proper embryos for transfer is a critical step in any IVF-treatment. The search for prognostic factors that predict embryo development and the outcome of IVF treatment have attracted considerable research attention as it is anticipated that knowledge of such factors may improve future IVF treatments. Current selection procedures are mostly entirely based on morphological evaluation of the embryo at different timepoints during development and particularly an evaluation at the time of transfer using a standard stereomicroscope. However, it is widely recognized that the evaluation procedure needs qualitative as well as quantitative improvements.

Reference is made to the following patent application disclosing culturing and imaging of cells as well as selection of embryos: WO 2004/056265, WO 2007/042044, WO2007/144001, WO 2009/003487, and WO 2010/003423. All patent and non-patent references cited in the application, or in the present application, are also hereby incorporated by reference in their entirety.

### SUMMARY OF THE INVENTION

A way to identify a viable embryo in a cohort of embryos from an IVF treatment would be to compare the recorded temporal pattern of cell division, represented by the morphokinetic parameters, to the recorded temporal patterns of cell division from embryos in past treatment cycles. A viable embryo would be characterized by having morphokinetic parameters that match the recorded morphokinetic parameters from embryos that implanted and resulted in a live birth in the past. In selecting the embryo for transfer that display morphokinetic parameters resembling those of positive embryos (i.e. embryos from ongoing or successfully completed pregnancies) and differ where possible from the majority of negative embryos (i.e. those embryos that failed to implant or gave rise to clinical abortions) it would be possible to improve the likelihood of obtaining a pregnancy and to achieve the desired outcome of the fertility treatment.

However, it is unlikely that selection criteria derived from morphokinetic parameters would be universally applicable as several factors have been shown to effect embryo development and the timing of cell divisions. The factors that have been shown to influence embryo development, and consequently the derived morphokinetic parameters, include: Temperature, media composition, pH, CO₂ and oxygen, growth factors, cultivation vessel etc. Other factors such as patient age, etiology, BMI, stimulation protocol (agonist/antagonist, type of hormone rFSH/hMG), embryo handling (pipettes, fertilization method, assisted hatching, removal of blastomeres, polar bodies or trophectoderm cells by biopsy) have been proposed by various scientists to influence embryo development and in particular the timing of cellular events such as cell cleavage. One purpose of the invention is therefore to utilize the global knowledge obtained from past embryo treatment cycles, however taking consideration to the local factors influencing embryo development, when establishing quality criteria for selection of optimal embryos to be implanted after in vitro fertilization (IVF). A first aspect of the invention therefore relates to a method for monitoring embryos being cultured under a first set of conditions, the method comprising the steps of:
a. providing
   i. a first embryo dataset for embryos that have been cultured and/or monitored under said first set of conditions, and
   ii. at least one second embryo dataset for embryos that have been cultured and/or monitored under at least a second set of conditions,
b. determining
   i. a first group of statistical parameters by analysing said first embryo dataset,
   ii. a second group of statistical parameters by analysing said at least one second embryo dataset, and
c. comparing the first group of statistical parameters to the second group of statistical parameters thereby detecting differences between the first and second groups of statistical parameters.

The present invention is most naturally applied to human embryos, but may also be applied within monitoring of any mammal embryos.

In a first embodiment the invention may be applied for determining, adapting and/or customizing embryo quality criteria for said embryos being cultured and/or monitored under said first set of condition. This may be applied by determining one or more embryo quality criteria by analysing a subset of said at least one second embryo dataset and adapting said embryo quality criteria to be applicable for the first set of conditions by comparing the first group of statistical parameters to the second group of statistical parameters. The obtained embryo quality measure may then be used for identifying and selecting embryos suitable for transplantation into the uterus of a female in order to provide a pregnancy and live-born baby. The obtained embryo quality measure may also be used for identifying and selecting embryos suitable for freezing and subsequent storing for possibly later thawing and transplantation.

In another embodiment of the invention the detected differences in the statistical parameters may be used to determine differences, i.e. differences in conditions, between the first set of conditions and the second set of conditions. The invention may then be applied within surveillance and monitoring of embryo development parameters and/or quality criteria to detect morphokinetic changes that may be caused by changes in the set of conditions where under the embryos are cultured and/or monitored, such as protocol, media, disposables or other protocol parameters that could ultimately affect the outcome. I.e. the present invention may be applied as quality control providing early warning of developmental problem.

The method according to the invention may be computer implemented or at least partly computer implemented thereby providing an efficient customizable tool for both experienced and less experienced fertility clinics. I.e. the method according to the invention may be implemented in automated incubators for culturing and monitoring embryos, such as human embryos. By implementing the present invention in such automated incubators, the selection processes, the quality control of e.g. culture media and other culturing conditions, adaptation of data between clinics and between different historical periods, may be more or less automated, i.e. fully manual with the software assisting the users with proposed decisions, semi-automatic or fully automatic with the incubator making all the decisions based on data analysis.

In a further aspect the invention relates to a system having means for carrying out the methods described above. Said system may be any suitable system, such as a computer comprising computer code portions constituting means for executing the methods as described above.

The system may further comprise means for acquiring images of the embryo at different time intervals, such as the system described in WO 2007/042044.

In a yet further aspect the invention relates to a data carrier comprising computer code portions constituting means for executing the methods as described above.

Definitions

An important improvement in embryo monitoring is the advent of time-lapse imaging. Time-lapse imaging throughout embryo development provide detailed information about the cellular events that take place during embryo development such as the timing of cell divisions (e.g. time and duration of cell cleavage, time interval between divisional events, synchrony of cleavage for sibling daughter cells etc.). All events may typically be expressed as hours post ICSI microinjection. Based on acquired time lapse image series a range of morphokinetic parameters can be defined, such as:

Cleavage times tN, denoted by the number of cells generated by the cell cleavage, e.g. t4 is the time of cell division to the four cell stage, i.e. the time of completion of the third cell division, etc. Cleavage time is defined as the first observed timepoint when the newly formed blastomeres are completely separated by confluent cell membranes. In the present context the times are expressed as hours post ICSI microinjection or post time for mixing of semen and oocyte in IVF, i.e. the time of insemination. This is the time of the deliberate introduction of sperm into the ovum. However, herein the term fertilization is also used to describe this timepoint. Thereby the cleavage times are as follows:
t2: Time of cleavage to 2 blastomere embryo
t3: Time of cleavage to 3 blastomere embryo
tn: Time of cleavage to n blastomere embryo

Cleavage period: The period of time from the first observation of indentations in the cell membrane (indicating onset of cytoplasmic cleavage) to the cytoplasmic cell cleavage is complete so that the blastomeres are completely separated by confluent cell membranes.

Duration of divisional stages, dN, numbered after the number of cells generated by the divisional event, d2, d4, d8, etc.

Duration of quiet stages qN. Interdivision periods with very little change in the position of cytoplasmic membranes (i.e. low blastomere activity). Named after the number of cells in the given period, q2, q4, q8.

Synchrony (cleavage of sister cells) sN,

One definition of the second synchrony s2, as the duration of the division from a 2 blastomere embryo to a 4 blastomere embryo s2=t4−t3, which corresponds to the duration of the period as 3 blastomere embryo. Similar definitions can be made for s3=t8−t5 etc. Synchronies may therefore be defined as follows:
s2=t4−t3: Synchrony in division from 2 blastomere embryo to 4 blastomere embryo.
s3=t8−t5: Synchrony in division from 4 blastomere embryo to 8 blastomere embryo.

Cell cycle time (DNA replication time) ccN. Time required to replicate DNA. One definition of the duration of the second cell cycle as the time from division to a two blastomere embryo until division to a 3 blastomere embryo cc2=t3−t2, i.e. the second cell cycle is the duration of the period as 2 blastomere embryo. The third cell cycle is cc3=t5−t3 etc. Duration of cell cycles may therefore be defined as follows:
cc1=t2: First cell cycle.
cc2=t3−t2: Second cell cycle, duration of period as 2 blastomere embryo.
cc3=t5−t3: Third cell cycle, duration of period as 3 and 4 blastomere embryo.
cc4=t9−t5: Fourth cell cycle, duration of period as 5-8 blastomere embryo.

See FIG. 1 for an illustration of an embryo cleavage pattern showing cleavage times (t2−t5), duration of cell cycles (cc1−cc3), and synchronies (s1−s3) in relation to images obtained.

Long cell cycle (Lcc) and Short cell cycle (Scc) are defined as embryos with an unusual long or short cell cycle, respectively. One definition of Lcc could be t2>32 hours and one definition of Scc could be cc2<5 hours. These criteria can be used as exclusion criteria to obtain a group of normal developing embryos (Medium cell cycle, Mcc).

Rearrangement of cellular position=Cellular movement (see below)

Cellular movement: Movement of the centre of the cell and the outer cell membrane. Internal movement of organelles within the cell is NOT cellular movement. The outer cell membrane is a dynamic structure, so the cell boundary will continually change position slightly. However, these slight fluctuations are not considered cellular movement. Cellular movement is when the centre of gravity for the cell and its position with respect to other cells change as well as when cells divide. Cellular movement can be quantified by calculating the difference between two consecutive digital images of the moving cell. An example of such quantification is described in detail in the PCT application WO 2007/042044 entitled “Determination of a change in a cell population”. However, other methods to determine movement of the cellular centre of gravity, and/or position of the cytoplasm membrane may be envisioned e.g. by using FertiMorph software (ImageHouse Medical, Copenhagen, Denmark) to semi-automatically outline the boundary of each blastomere in consecutive optical transects through an embryo.

Organelle movement: Movement of internal organelles and organelle membranes within the embryo which may be visible by microscopy. Organelle movement is not cellular movement in the context of this application.

Movement: spatial rearrangement of objects. Movements are characterized and/or quantified and/or described by many different parameters including but restricted to: extent of movement, area and/or volume involved in movement, rotation, translation vectors, orientation of movement, speed of movement, resizing, inflation/deflation etc. Different measurements of cellular or organelle movement may thus be used for different purposes some of these reflect the extent or magnitude of movement, some the spatial distribution of moving objects, some the trajectories or volumes being afflicted by the movement.

The embryo quality criteria may be the earlier stage quality criteria as disclosed in WO 2007/144001 and in pending PCT application PCT/DK2012/05018 entitled “Embryo quality assessment based on blastomere cleavage and morphology” filed at May 31, 2012, and it may be the later blastocyst related criteria as disclosed in the pending application U.S. 61/663,856 entitled “Embryo quality assessment based on blastocyst development” filed at Jun. 25, 2012. These applications are therefore also hereby incorporated by reference in their entirety.

Embryo quality is a measure of the ability of said embryo to successfully implant and develop in the uterus after transfer. Embryos of high quality will most likely successfully implant and develop in the uterus after transfer whereas low quality embryos will most likely not develop.

Embryo quality criteria (or selection criteria) are a set of parameters relating to the quality of the embryo. Embryo quality criteria are directly related to and provide the basis for choosing embryo selection criteria.

Embryo viability is a measure of the ability of said embryo to successfully implant and develop in the uterus after transfer. Embryos of high viability will most likely successfully implant and develop in the uterus after transfer whereas low viability embryos will most likely not develop. Viability and quality are used interchangeably in this document

Embryo quality (or viability) measurement is a parameter intended to reflect the quality (or viability) of an embryo such that embryos with high values of the quality parameter have a high probability of being of high quality (or viability), and low probability of being low quality (or viability). Whereas embryos with an associated low value for the quality (or viability) parameter only have a low probability of having a high quality (or viability) and a high probability of being low quality (or viability).

### DRAWINGS

FIG. 1. Nomenclature for the cleavage pattern showing cleavage times (t2−t5), duration of cell cycles (cc1−cc3), and synchronies (s1−s3) in relation to images obtained.

FIG. 2. Variation of morphokinetic parameters (in this case t2, t3 and t5) as a function of the culture medium in a fertility clinic.

FIG. 3a. Schematic hierarchical decision tree with the parameters t5, s2 and cc2.

FIG. 3b. Example of embryo selection in a hierarchical decision tree with the parameters t5, s2 and cc2.

FIG. 3c. A series of images showing where the time of t2 (time of cleavage where a 2 blastomere embryo is created, i.e. the time of resolution of the cell division) is seen to happen at 22.9 hours.

FIG. 3d. A series of images showing direct cleavage to a 3 blastomere embryo. Cleavage from 1 to 3 cells happens in one frame, thus t3=t2.

FIG. 4. Percentage of embryos having completed a cell division by a given time after fertilization.

FIG. 5. Implantation rate in high and low implantation groups for the parameters t2, t3, t4, t5, cc2, cc3, and s2.

FIG. 6. Distribution of the timing for cell division to five cells, t5, for 61 implanting embryos (positive, blue dots) and for 186 non-implanting embryos (negative, red dots).

FIGS. 7a-7c. Percentage of implanting embryos with cell division times inside or outside ranges defined by quartile limits for the total dataset.

FIG. 8a-8b. Percentage of implanting embryos with cell division parameters below or above the median values.

FIGS. 9 to 25 show screen dumps from the applicant's EmbryoViewer wherein one or more of the methods according to the present invention have been implemented.

FIG. 9. An overview of time-lapse images of twelve embryos (horizontal) from the same woman with the embryo development over time (vertical).

FIG. 10. A close up of a single embryo with some of its morphokinetic parameters indicated to the right in the figure.

FIG. 11. A close up of three embryos with some of the morphokinetic parameters indicated below each embryo for comparison.

FIG. 12. Four embryos selected by the software based on hierarchical selection criteria and a certain selection algorithm. External selection criteria can be imported and adapted to the local selection criteria by means of the present invention.

FIG. 13. Four embryos selected by the software based on weighted average selection criteria and a certain selection algorithm. External selection criteria can be imported and adapted to the local selection criteria by means of the present invention.

FIG. 14a. Laboratory data for the twelve embryos indicating where the high quality embryos are located in the embryo micro-well holder and providing an overview of which embryos to transfer, freeze and discard.

FIG. 14b. Instrument data providing information of embryo culturing conditions.

FIG. 14c. Patient information providing an overview of the twelve embryos.

FIG. 15. Overview of pregnancy rates for good prognosis embryos that were implanted.

FIG. 16. Overview of morphokinetic parameters for all embryos in the database.

FIG. 17. Overview of morphokinetic parameters for ongoing embryos in the database, i.e. a functional subgroup of the embryos shown in FIG. 16.

FIG. 18. Overview of morphokinetic parameters for failed embryos in the database, i.e. a functional subgroup of the embryos shown in FIG. 16.

FIG. 19. Timings for t2, t3 and t5 (upper plot), cc2 (middle plot) and S2 (lower plot) for a selection of embryos (July 2009 to May 2011). Abrupt changes in the timing parameters might indicate a change in the culturing/monitoring conditions.

FIG. 20. Overview of embryos providing status, slide ID, well no., and various morphokinetic parameters for each embryo. In the bottom various statistical parameters are provided for the entire shown collection of embryos.

FIG. 21. Statistical distributions (accumulated) for morphokinetic parameters (t2, t3, t4, t5, cc2 and s2) compared for different embryo datasets: a historical dataset for 2010 and most recent data since January 2011.

FIG. 22. Distributions of morphokinetic parameters (t2, t3, t4, t5, cc2 and s2) compared for different embryo datasets: a historical dataset for 2010 and most recent data since January 2011.

FIG. 23. Statistical distributions (ratios) for morphokinetic parameters (t2, t3, t4, t5, cc2 and s2) compared for different embryo datasets: a historical dataset for 2010 and most recent data since January 2011.

FIG. 24. Statistical distributions for morphokinetic parameters (t2, t3, t4, t5, cc2 and s2) compared for different embryo datasets: a historical dataset for 2010 and most recent data since January 2011. As seen FIGS. 21-24 provide different tools for overview and comparison between datasets in order for a user of the software to be able distinguish and survey the development in culturing and monitoring conditions of the embryo, i.e. quality control.

FIG. 25. Three graphs showing different embryo success rates over time (time along x-axis). The top graph shows fertilization and implantation rates with respect to number of treatments with transfer, the middle graph shows hCG, gestational sacs and liveborn babies with respect to number of treatments with transfer and the bottom graph shows transfer and freeze rates with respect to number of photographed wells. Thus, the different embryo success rates can be monitored over time to provide quality control.

FIG. 26. Statistical distributions for timing of cell divisions t2, t3, t4 and t5 with data originating from two different fertility clinics (see example 2).

FIG. 27. Statistical distributions for cell division parameters cc2, cc3, s2 and s3 with data originating from two different fertility clinics (see example 2).

FIG. 28. Mouse embryo development with varying temperature of the incubation medium (see example 3).

FIG. 29. Duration between various cell divisions for mouse embryos for varying temperatures of the incubation medium (see example 3).

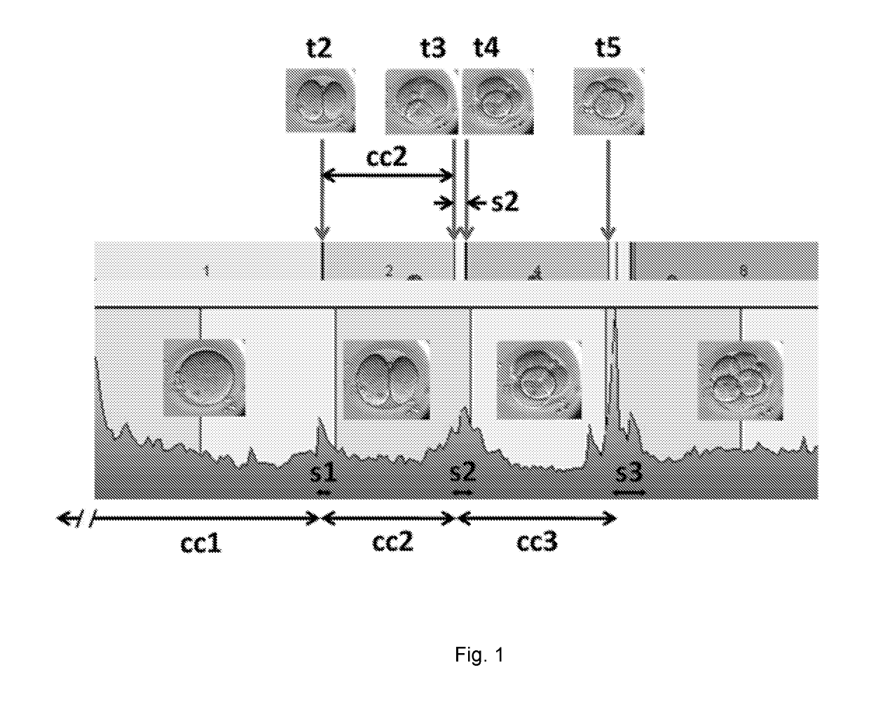

### DETAILED DESCRIPTION OF THE INVENTION

One embodiment of the present invention addresses the problem of directly adapting selection criteria from one fertility clinic to another. When several factors have been shown to effect embryo development a direct adaptation of selection criteria may require an exact replication of the treatment protocol and an assumption that the patient groups are identical (age, etiology, etc). As this is highly unlikely direct adaptation of selection criteria may lead to non-optimal embryo selection with a likely inferior outcome.

The present invention also addresses the challenges for a novel fertility clinic to collect sufficient time-lapse data from embryos with known positive implantation to determine their own distinctive morphokinetic quality markers (e.g. suitable selection/quality criteria based on morphokinetic parameters) and to start optimizing their selection criteria. The present invention is therefore highly beneficial for the novel fertility clinic to be able to use the selection criteria derived by one or more experienced fertility clinics based on their extensive dataset.

In one embodiment of the invention differences in conditions between the first set of conditions and the second set of conditions are determined based on the detected differences between the first and second group of statistical parameters.

In a further embodiment of the invention one or more embryo quality criteria are determined by analysing a subset of said at least one second embryo dataset. And furthermore said embryo quality criteria derived from the subset of the second embryo dataset may be adapted to be applicable for the first set of conditions based on comparing the first group of statistical parameters to the second group of statistical parameters.

In a further embodiment of the invention one or more embryo quality criteria are determined by analysing a subset of said first embryo dataset. And preferably the embryo quality criteria extracted from the first embryo dataset are the same type of embryo quality criteria extracted from the subset of the second embryo dataset. The invention may thereby also apply to the situation where the inexperienced clinic begins to compile sufficient data to develop their own quality criteria, which can then be taken into account when adapting the quality criteria extracted from the second embryo dataset (e.g. from the experienced clinic). An iterative adaptation between own embryo quality criteria and external embryos quality criteria is thereby obtained.

In a further embodiment of the invention the subset(s) of an embryo dataset comprise preimplantation data from implanted embryos that have resulted in ongoing pregnancies, live born babies, fetal heart beat (FHB), and/or gestational sacs. I.e. the subset is selected to reflect high quality embryos with proven track record.

The statistical parameters may be any combination of known statistical parameters, such as mean, median, quartiles, standard deviation, ranges(min-max), percentiles, variance, etc. The types of the statistical parameters in the first and second group of statistical parameters preferably correspond to each other such that they are comparable.

In yet another embodiment of an embryo dataset (e.g. a first or second embryo dataset) comprise morphokinetic parameters for

1) all embryos in a group of monitored embryos, or

2) a functionally defined subgroup from the group of embryos.

I.e. all embryos in group of monitored embryos (i.e. all embryos ever monitored in a certain clinic) can be selected as the frame of reference for the statistical calculations. Or just a subgroup is selected where this subgroup is functionally defined. Examples of functionally defined subgroups:
all fertilized embryos in the group,
embryos that have divided to at least a predefined number of cells at a predefined number of hours after insemination, such as divided to at least 7 cells 68 hours after insemination,
embryos that have less than a predefined percentage of fragmentation at a predefined hours after insemination, e.g. less than 20% fragmentation 68 hours after insemination,
embryos that are not multinucleated at a certain cell stage, e.g. at the four cell stage,
embryos that have been classified as “Good quality embryos” (GQE) by a qualified embryologist,
embryos that have been chosen for freeze or transfer,
embryos that have been chosen for transfer, and/or
embryos that have implanted.
Embryos selected by excluding poorly developing embryos, e.g. by excluding Scc and/or Lcc embryos or by employing other exclusion criteria as e.g. described in pending applications PCT/DK2012/05018 or U.S. 61/663,856, the latter entitled “Embryo quality assessment based on blastocyst development”.

In a further embodiment of the invention the morphokinetic parameters are selected from the group of:
the timing and/or duration cell-division periods and inter-division periods,
the timing and/or duration of: cleavage times, cleavage periods and cell cycle times;
the timing and/or duration of divisional stages and quiet stages,
synchrony of cell divisions;
timing, extent or duration of cellular and/or organelle movement,
timing, extent or duration of quality criteria, such as quality criteria as described in PCT/DK2012/05018
Blastocyst quality criteria as described in U.S. 61/663,856

In a further embodiment of the invention the morphokinetic parameters are selected from the group of:
the timing and/or duration cell-division periods and inter-division periods, determined for the first, second, third, fourth, fifth and/or sixth cell division;
the timing and/or duration of: cleavage times, cleavage periods and cell cycle times determined for the first, second, third, fourth, fifth and/or sixth cell division;
the timing and/or duration of divisional stages and quiet stages determined for the first, second, third, fourth, fifth and/or sixth cell division;
synchrony of the second and third cell division;
timing, extent or duration of cellular and/or organelle movement determined for the first, second, third, fourth, fifth and/or sixth cell division;
timing, extent or duration of cellular and/or organelle movement determined in between the first, second, third, fourth, fifth and/or sixth cell division;

In a further embodiment of the invention said one or more embryo quality criteria extracted from the second embryo dataset is selected from the group of:
embryo quality criteria validated by additional datasets,
embryo quality criteria validated by retrospective studies,
embryo quality criteria validated by prospective studies,
embryo quality criteria validated by resampling, and/or
embryo quality criteria validated by bootstrapping.

One of the aims of the present invention is to apply “global” embryo quality parameters to “local” embryo quality parameters with the goal of raising the quality of the local embryo selection criteria, however taking considerations to the “local” conditions. The different sets of culturing and monitoring conditions for the embryos then apply to the conditions in “local” and “global”.

“Local” and “global” can apply to many situations. Local may be the novice fertility clinic with only few embryo data and global may be an external fertility clinic with an immense embryo data collection. But “local” and “global” may also to apply different culturing devices in the same locality. Thus:

In one embodiment of the invention the first set of conditions corresponds to the conditions in a first fertility clinic (such as a local fertility clinic). Thus, the first embryo dataset may originate from a local fertility clinic.

In a further embodiment of the invention the second set of conditions corresponds to the conditions in second fertility clinic (such as an external fertility clinic). Thus, a second embryo dataset may originate from an external fertility clinic.

In a further embodiment of the invention the first and second set of conditions correspond, respectively, to the conditions in two different devices for culturing and/or monitoring embryos. Thus, the first and second embryo datasets originate, respectively, from two different devices for culturing and/or monitoring embryos. The two different devices may be at the same or different localities.

In a further embodiment of the invention said first and second embryo datasets originate from the same locality wherein the first embryo dataset comprise the most recent embryo data and the second embryo dataset comprise older historical embryo data. E.g. the first and second sets of conditions correspond to the conditions in one device for culturing and/or monitoring embryos before and after, respectively, the culture medium was changed.

In a further embodiment of the invention said first embryo dataset is substantially smaller than the second embryo dataset, such as 2 times smaller, such as 5 times smaller, such as 10 times smaller, such as 50 times smaller, such as 100 times smaller, such as 200 times smaller, such as 500 times smaller, such as 1000 times smaller.

In a further embodiment of the invention the embryos are cultured and/or monitored in an incubator. Preferably the embryos are monitored through image acquisition, e.g. by means of time-lapse microscopy equipment, such as image acquisition at least once per hour, preferably image acquisition at least once per half hour such as image acquisition at least once per twenty minutes, such as image acquisition at least once per fifteen minutes, such as image acquisition at least once per ten minutes, such as image acquisition at least once per five minutes, such as image acquisition at least once per two minutes, such as image acquisition at least once per minute.

One embodiment of the present invention describes a method to adapt embryo selection criteria based on morphokinetic parameters derived from time-lapse imaging from one clinic, the “experienced” clinic, to the protocols and incubation conditions in another clinic, the “novice” clinic. A further embodiment of the invention relates to an iterative procedure to continually improve selection criteria within the novice clinic by:
i) inclusion of novel data from procedures with known outcome performed by the novice clinic
ii) incorporating data from additional more experienced clinics, and
iii) empirically determine specialized selection criteria for subgroups of patients with special etiology or needing special laboratory procedures (ICSI, PGD etc.).

In a fertility treatment ovarian hyper stimulation causes maturation of numerous oocytes in a single stimulation cycle. Most treatment cycles lead to retrieval of 6 to 20 oocytes (typically 8 to 12). A few of these oocytes will normally fail to fertilize (not 2PN's) or fail to develop through the first cleavage cycle. However, most IVF treatment cycles still give many cleavage stage embryos that could be transferred back to the uterus of the patient, but only a single or two embryos are selected for transfer in a typical treatment cycles. Most fertility cycles fail to produce the desired pregnancy (clinical pregnancy rate in DK 2010 was 30% per cycle with transfer), and in case of dual embryo transfer (still the most common procedure in DK and the US) not all embryos may implant. Only in those treatments where the number of implanted embryos matches the number of transferred embryos it can be assumed to know, which embryos that implanted (ignoring monozygotic twinning) and the embryos with known positive implantation are therefore a small minority of the total number of embryos handled—even in the best and most experienced clinics.

Experienced user of time-lapse imaging having data from 1000 treatment cycles with retrieval of 10 embryos in each cycle of which 60% develop to cleavage stage. This clinic would have time lapse images and morphokinetic parameters for about 6000 cleavage stage embryos. Assuming on the average 1.8 embryo were chosen for transfer per cycle (i.e. 1800 embryos), it is still only expected that 33% of the cycles lead to ongoing pregnancy (i.e. 600 embryos). Most pregnancies with dual embryo transfer were likely to be singleton pregnancies, where it cannot be safely assumed which embryo implanted. In the end the clinic would end up with less than 300 embryos where they knew there was an ongoing implantation and about 1200 embryos that failed to implant. For the large majority (i.e. 4500) of the embryos they would not know if they were viable or not.

Novice user of time-lapse imaging having data from 50 treatment cycles with retrieval of 10 embryos in each cycle of which 60% develop to cleavage stage. This clinic would only have time lapse images and morphokinetic parameters for about 300 cleavage stage embryos. Assuming on the average 1.8 embryo were chosen for transfer per cycle (i.e. 90 embryos), they would most likely end up with only 15 embryos where they knew there was an ongoing implantation and about 75 embryos that failed to implant.

For the large majority (i.e. 210) of the embryos they would not know if they were viable or not.

A similar problem is presented when attempting to derive specialized morphokinetic selection criteria for small subgroups of patients (PCOS patients, advanced maternal age, endometriosis etc.) whose embryos may develop differently either due to the source etiology or because of an unusual stimulation protocol that may be required to treat these patients (low stimulation for PCOS, high stimulation for low ovarian reserve etc.). In these cases not even the largest clinics may have enough data from comparable IVF cycles to derive specialized criteria. In these cases it would be highly beneficial to be able to combine data from many different clinics to obtain a sufficiently large dataset. However, to evaluate the combined dataset it is necessary to take into consideration the effect of small differences in protocol between the clinics and to correct for these differences in order to derive generally applicable selection criteria. The present invention addresses this problem.

In a further embodiment of the invention the selection criteria in a given clinic are iteratively improved by incorporating information from implanting and failed embryos from recent cycles. This ongoing iteratively improvement and refinement of the selection criteria will advantageously lead to:
a) Improved understanding of embryology, and the importance of the different morphokinetic parameters
b) Improved success rates
c) Improved communication to the patient about why a treatment failed and when other methods (e.g. adoption) should be considered)
d) Consequently reducing costs for the clinic, the patient and the society

Quality Control

A further embodiment of the invention applies within quality control in a clinic by comparing average cleavage patterns (morphokinetic parameters) of embryos in recent treatment cycles with cleavage patterns (morphokinetic parameters) from past cycles. Temporal changes in general morphokinetic parameters for Good Quality Embryos (as exemplified above) may indicate an unintended change in protocol, such as bad lot of media, problems with incubators, pipette tips, etc.

Constant monitoring of morphokinetic parameters are thus important for quality control and will be able to give early warnings for unintended differences in embryo handling. Morphokinetic parameter analysis may also be used to alleviate fears after multiple implantation failures that embryo development is indeed normal.

Detailed Description of Drawings

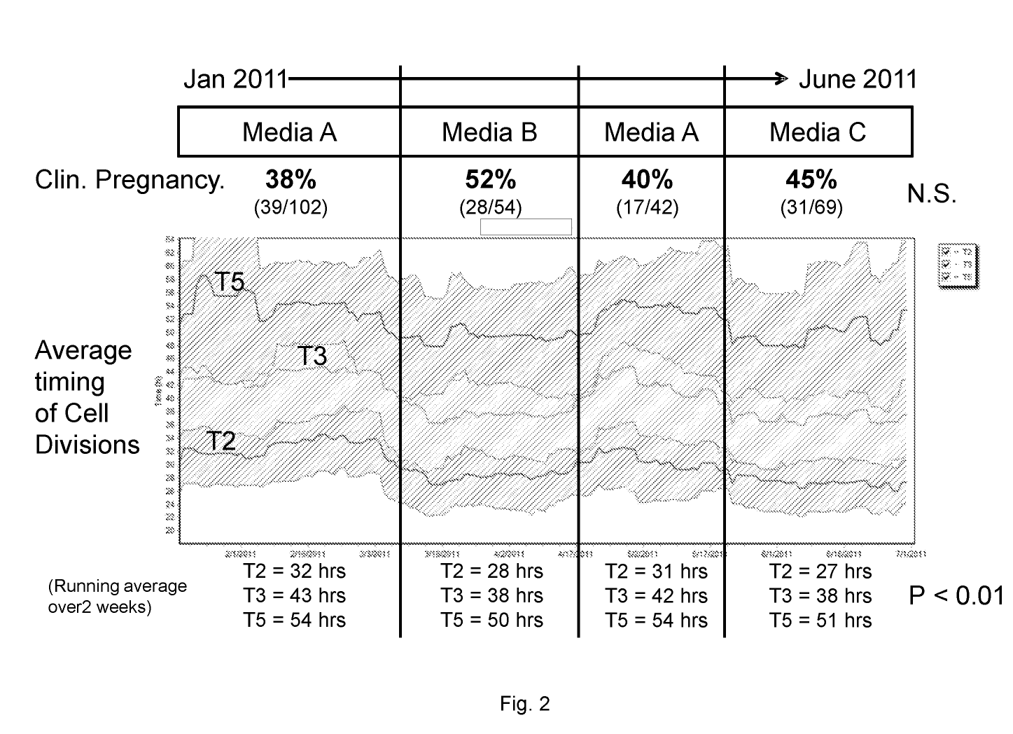

FIG. 2 shows the variation of morphokinetic parameters (in this case t2, t3 and t5) as a function of the culture medium in a fertility clinic. The total period runs from February 2011 to June 2011. Of the three media used (A, B, C) media A provided the worst embryo development (latest cell division timing and t2, t3 and t5 are all higher for media A). Media A also provided worse implantation rates and pregnancy rates. Media B and Media C both provided normal embryo development and high implantation and pregnancy rates. Applying the present invention to surveillance of morphokinetic parameters of embryos developing in different media can reveal these problems online as they progress.

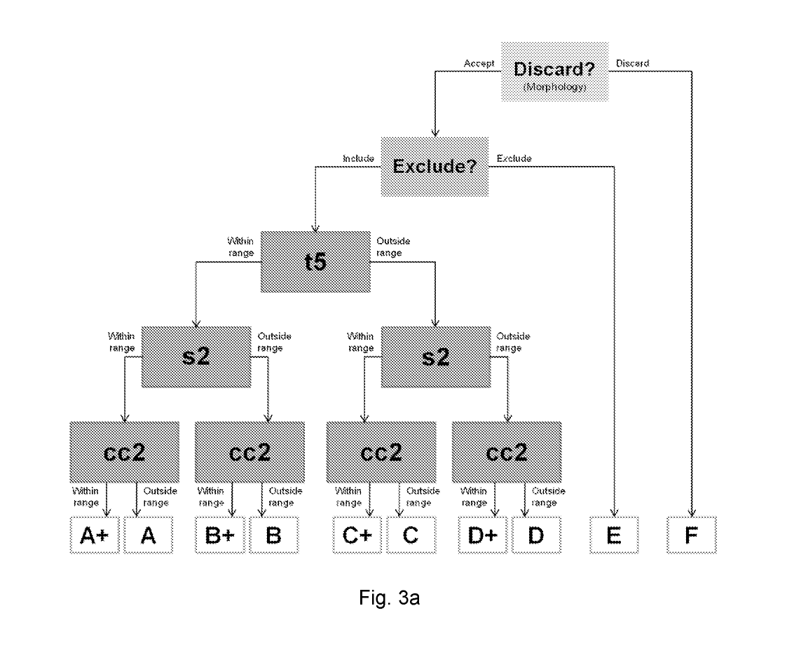

FIG. 3a shows a schematic hierarchical decision tree with the morphokinetic parameters t5, s2 and cc2 based on:
1. Morphological screening;
2. absence of exclusion criteria;
3. timing of cell division to five cells (t5);
4. synchrony of divisions from 2-cell to 4-cell stage, s2, i.e. duration of 3-cell stage;
5. duration of second cell cycle, cc2, i.e. time between division to 3-cell stage and division to 5-cell stage.

The classification generates ten grades of embryos with increasing expected implantation potential (right to left), i.e. A+ has highest expected implantation rate.

The decision tree depicted in FIG. 3a represents a sequential application of the identified selection criteria in combination with traditional morphological evaluation. In the decision tree in FIG. 3a embryos are subdivided into 6 categories from A to F. Four of these categories (A to D) are further subdivided into two sub-categories (+) or (−) as giving a total of 10 categories. The hierarchical decision procedure starts with a morphological screening of all embryos in a cohort to eliminate those embryos that are clearly NOT viable (i.e. highly abnormal, attretic or clearly arrested embryos). Those embryos that are clearly not viable are discarded and not considered for transfer (category F). Next step in the model is to exclude embryos that fulfil any of the three exclusion criteria: i) uneven blastomere size at the 2 cell stage, ii) abrupt division from one to three or more cells; or iii) multi-nucleation at the four cell stage (category E). Any of the exclusion criteria may be applied to each and every embryo monitored, or the embryo population may be subjected to exclusion criteria before applying the selection criteria. Exclusion criteria may include information of blastomere evenness at t2, information of multinuclearity at four-blastomere stage, and/or information of cleavage from one blastomere directly to three blastomeres.

The subsequent levels in the decision tree model follow a strict hierarchy based on the binary timing variables t5, s2 and cc2. An example is shown in FIG. 3b where 196 embryos (after exclusion of a number of embryos based on exclusion criteria) are placed into 8 categories based on the measured values of t5, s2 and cc2 and the chosen selection criteria.

First, if the value of t5 falls inside the optimal range (between 49.39 and 56.48 hours after insemination) the embryo is categorized as A or B. If the value of t5 falls outside the optimal range (or if t5 has not yet been observed at 64 hours) the embryo is categorized as C or D.

Second, if the value of s2 falls inside the optimal range (≦0.75 hours) the embryo is categorized as A or C depending on the measured value of t5 and similarly if the value of s2 falls outside the optimal range the embryo is categorized as B or D depending on t5.

Thirdly, the embryo is categorized with the extra plus (+) if the value for cc2 is inside the optimal range 12.0 hours) (A+/B+/C+/D+) and is categorized as A,B,C or D if the value for cc2 is outside the optimal range.

The depicted decision procedure thereby divides all the 196 evaluated embryos in eight different categories containing between 15 and 35 transferred embryos but with largely decreasing implantation potential (i.e. from 70% for A+ to 13% for D). This hierarchical decision procedure is a powerful tool when estimating and grading the development potential of a cohort of embryos but the example shows that it can be crucial to know the morphokinetic parameters and their statistical distribution under the specific set of culturing and monitoring conditions, because small changes in the culturing/monitoring conditions might result in changes of the observed morphokinetic parameters. And even small changes in the distribution of the morphokinetic parameters might provide faulty selection criteria in the depicted hierarchical decision tree.

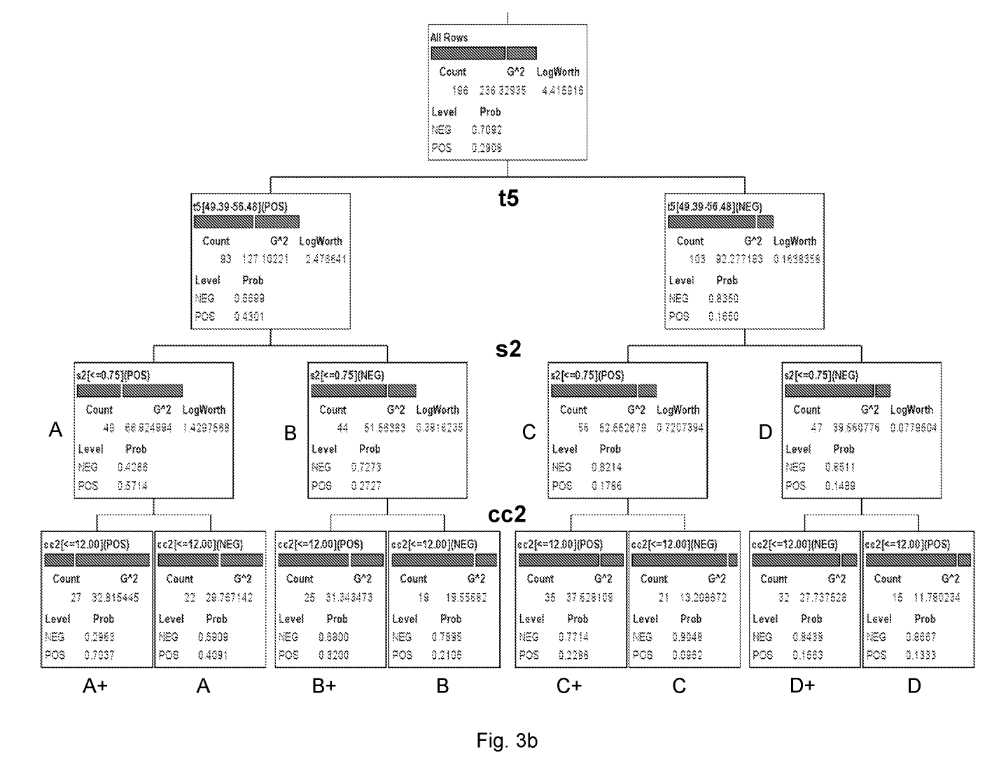

FIG. 4 shows the percentage of embryos having completed a cell division by a given time after fertilization. The steep blue curves represent implanting embryos, red curves (less steep) rpresent embryos that do not implant. Four curves of each color (i.e. four steep curves and four curves that are less steep) represent completion of the four consecutive cell divisions from one to five cells i.e. t2, t3, t4, and t5.

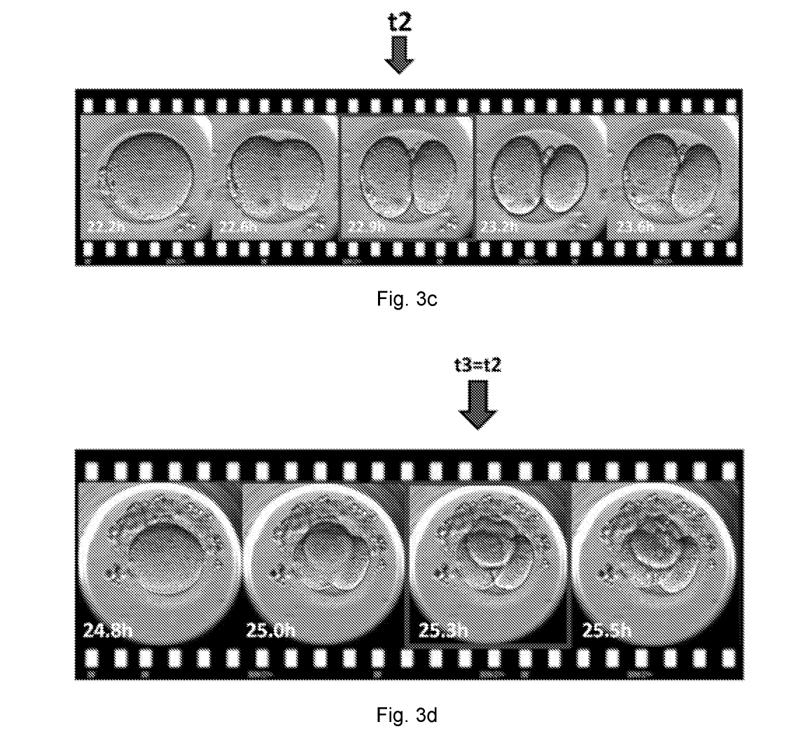

FIG. 5 shows implantation rate in high and low implantation groups for the parameters t2, t3, t4, t5, cc2, cc3, and s2.

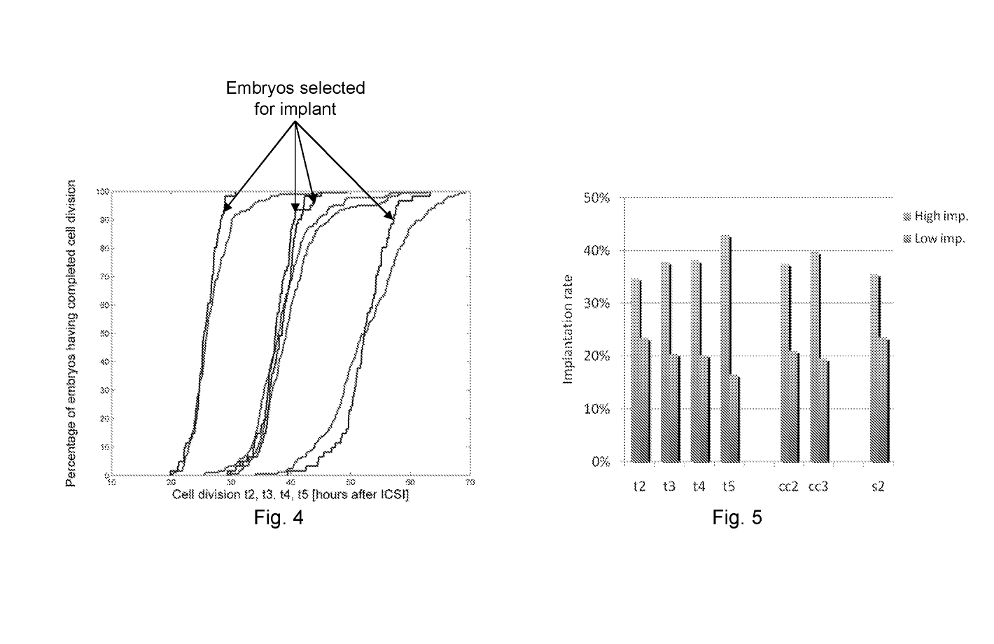

FIG. 6 shows the distribution of the timing for cell division to five cells, t5, for 61 implanting embryos (marked “POS” for positive) and for 186 non-implanting embryos (marked “NEG” for negative). The left panel show the overall distributions of cleavage times. The short horizontal lines demarcate standard deviations, means and 95% confidence limits for the mean. The boxes denote the quartiles for each class of embryos. The right panel shows the distribution of observed t5 cleavage times for the two types of embryos plotted as normal quartiles on a plot where a normal distribution is represented by a straight line. The two fitted lines represent normal distributions corresponding to the two types of embryos.

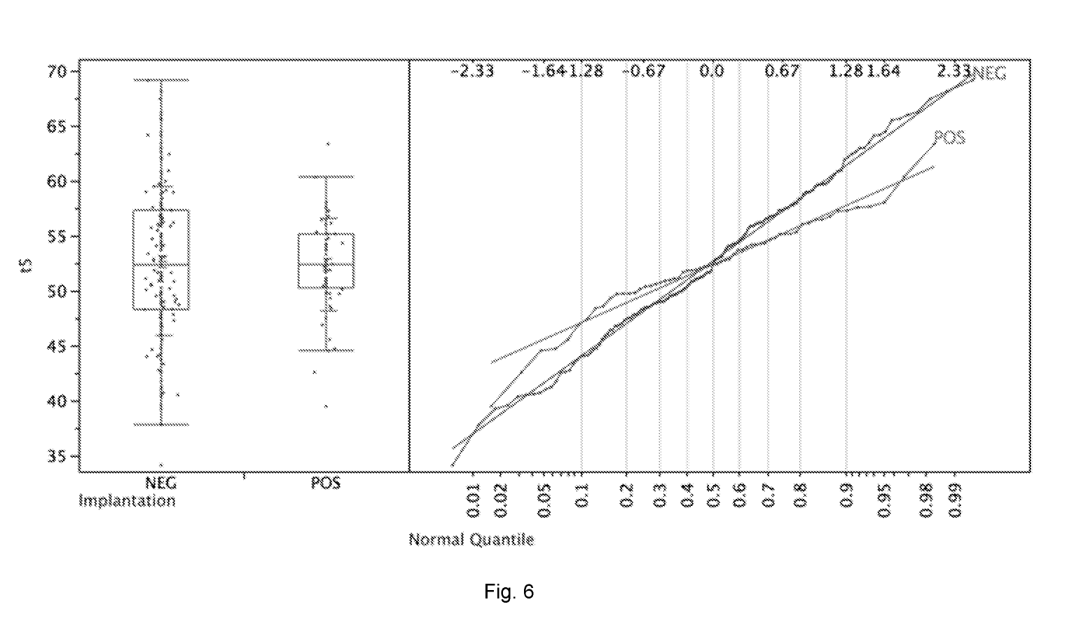

FIGS. 7a-7c show the percentage of implanting embryos with cell division times inside or outside ranges defined by quartile limits for the total dataset. The three figures show ranges and implantation rate for: division to 2-cells (t2) in FIG. 7a, division to 3-cells (t3) in FIG. 7b and division to 5-cells (t5) in FIG. 7c. As the limits for the ranges were defined as quartiles, each column represent the same number of transferred embryos with known implantation outcome, but the frequency of implantation was significantly higher for embryos within the ranges as opposed to those outside the ranges.

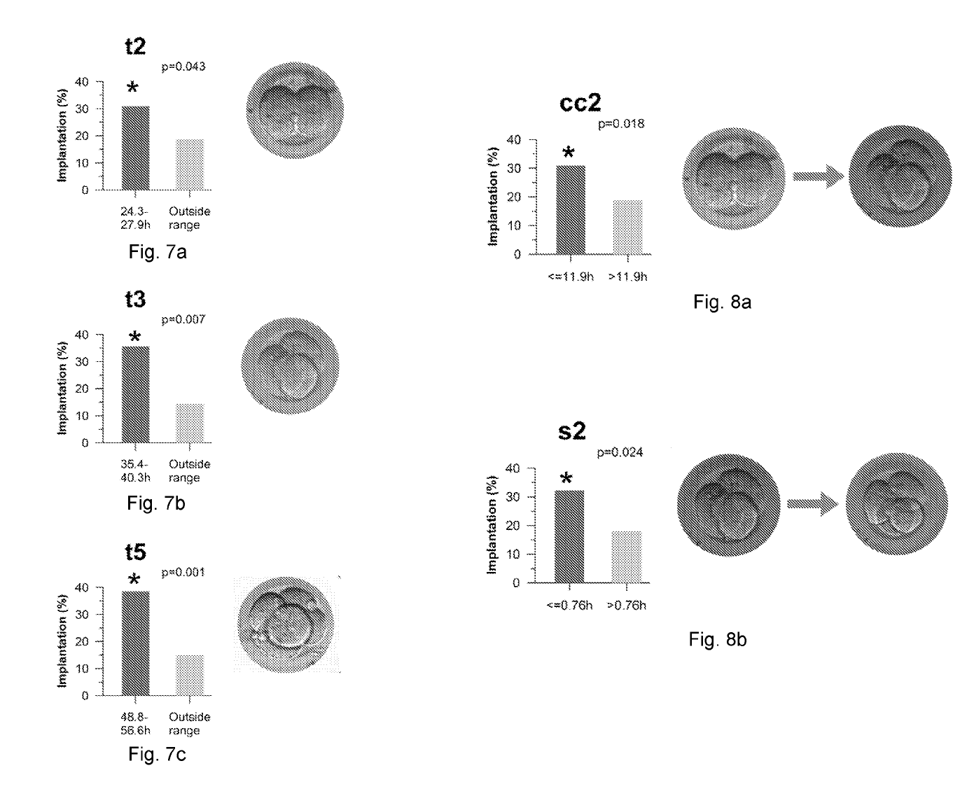

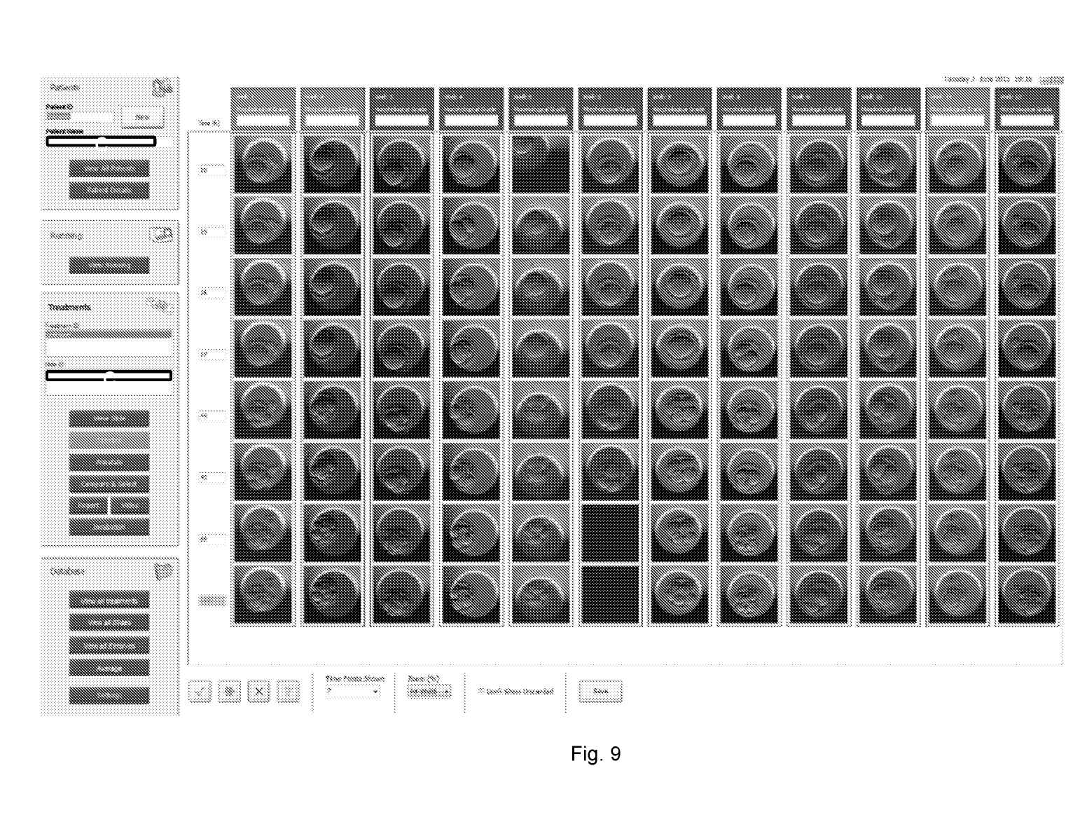

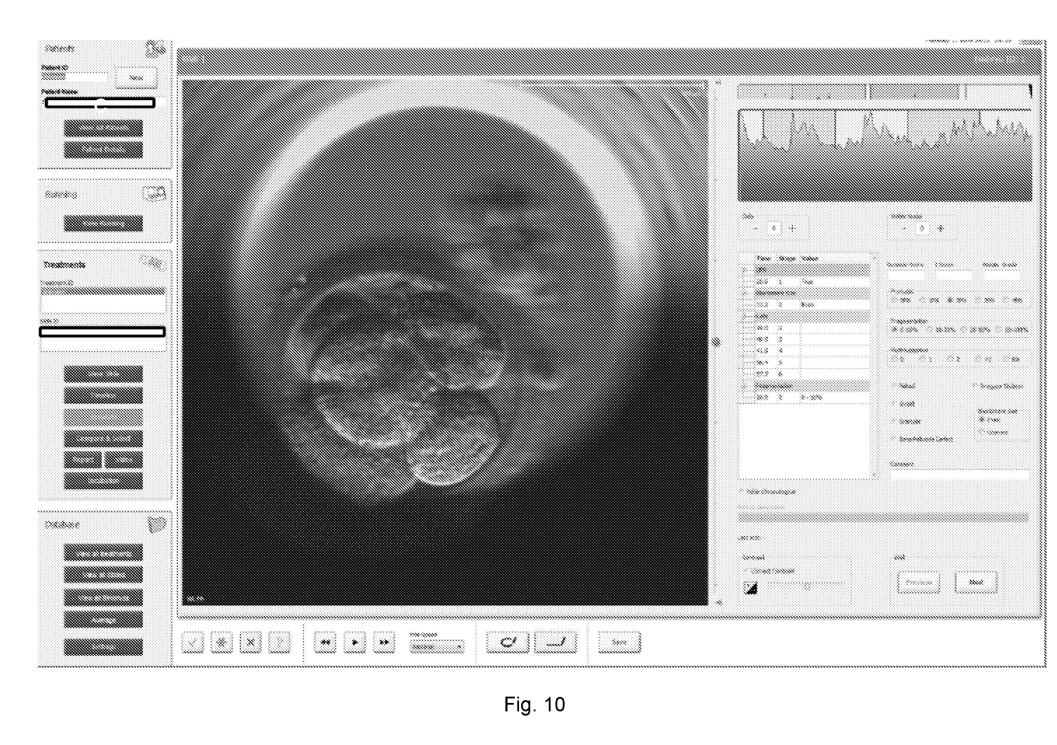

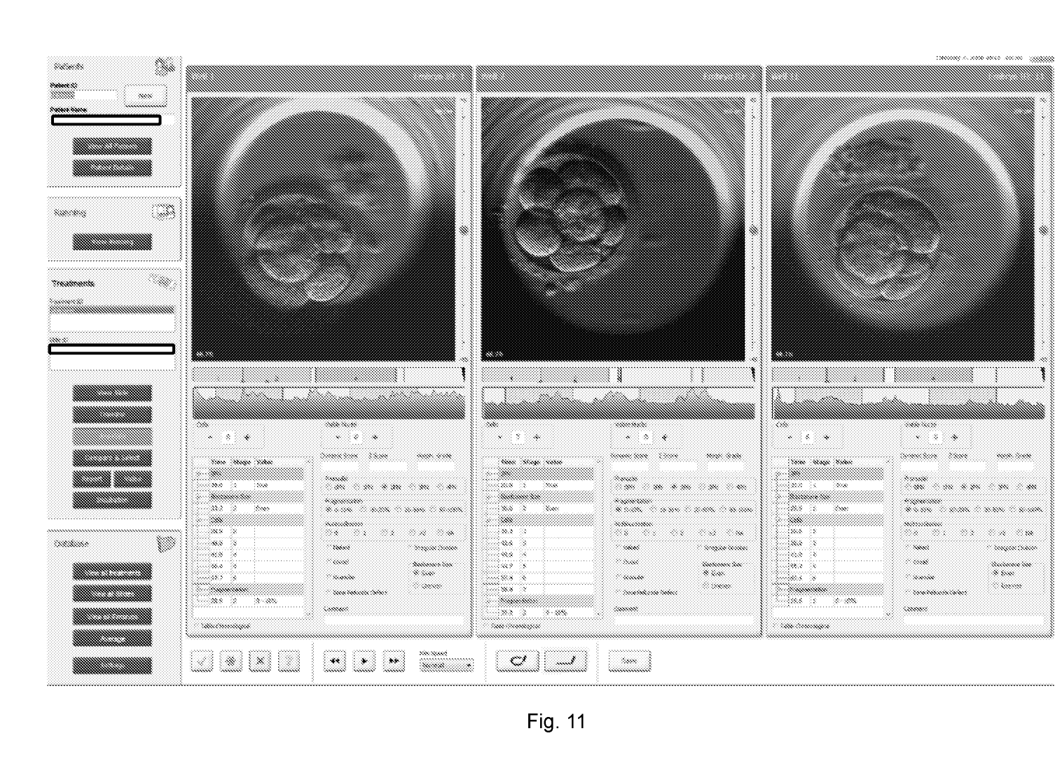

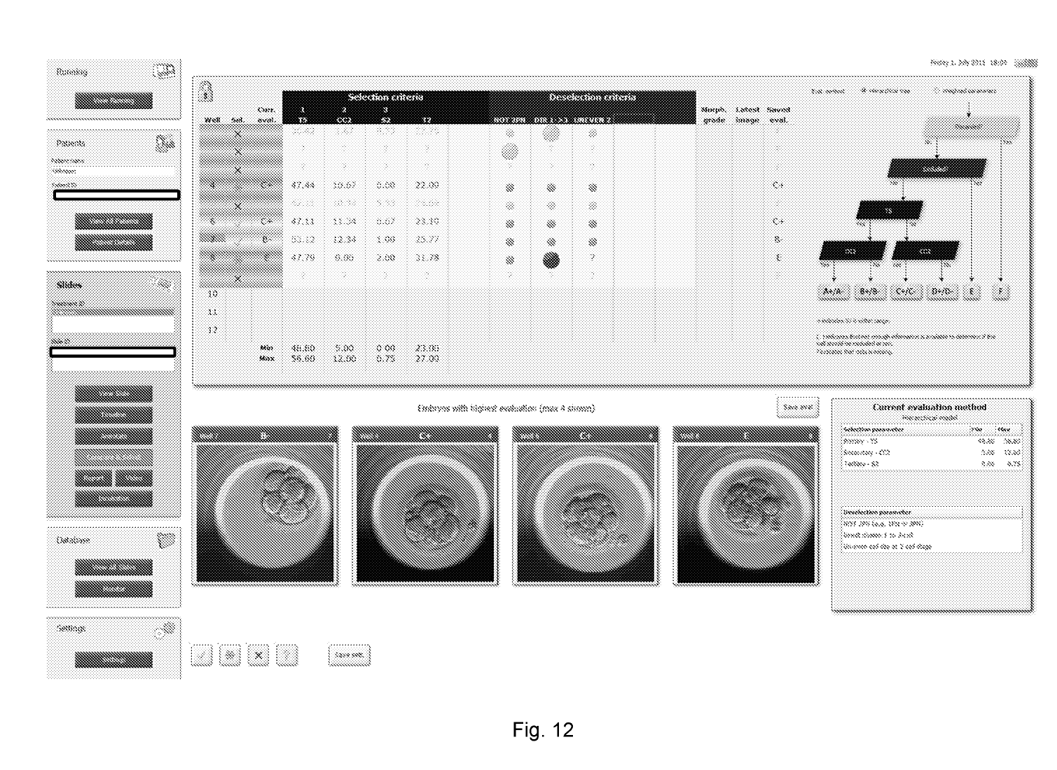

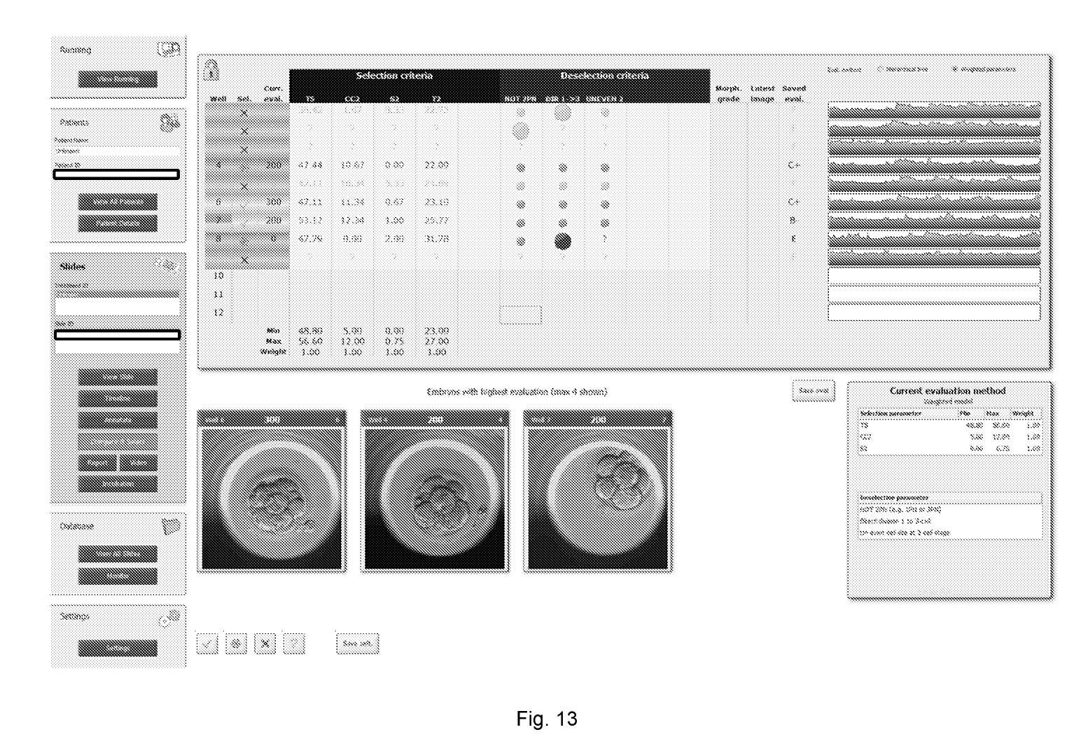

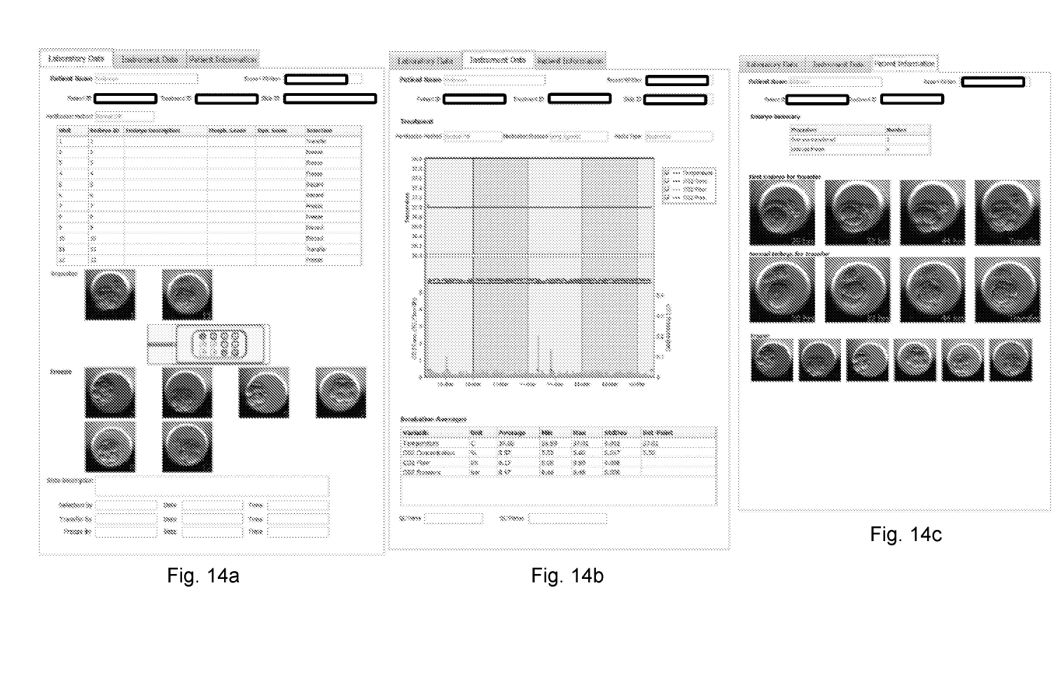

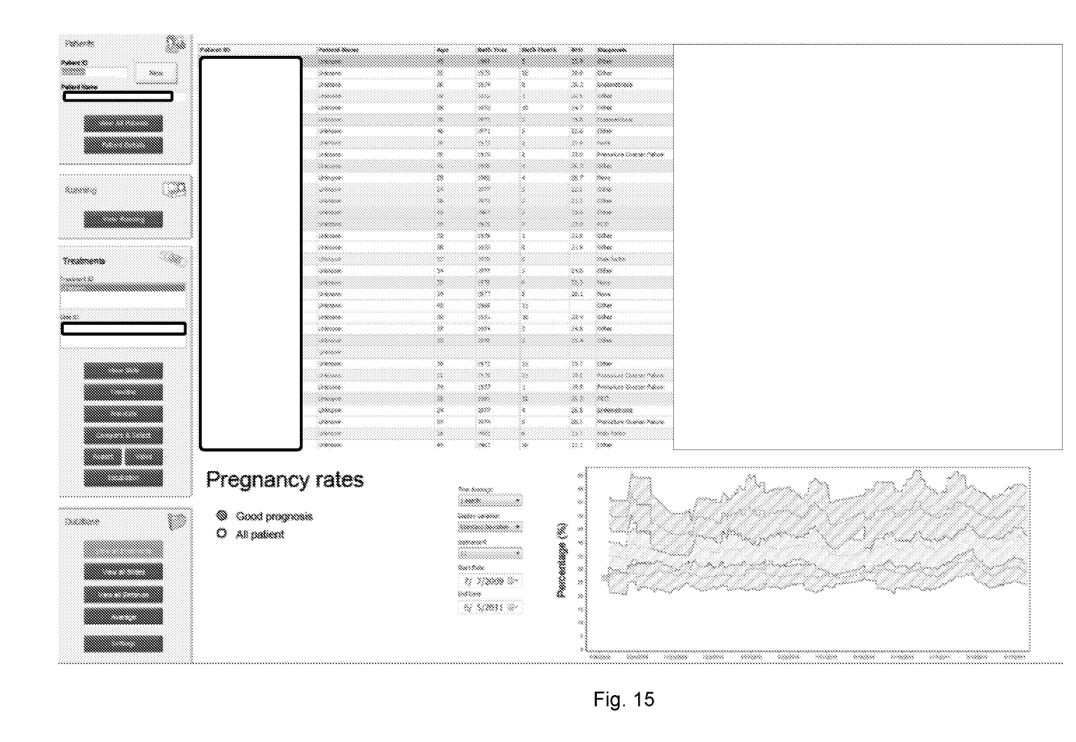

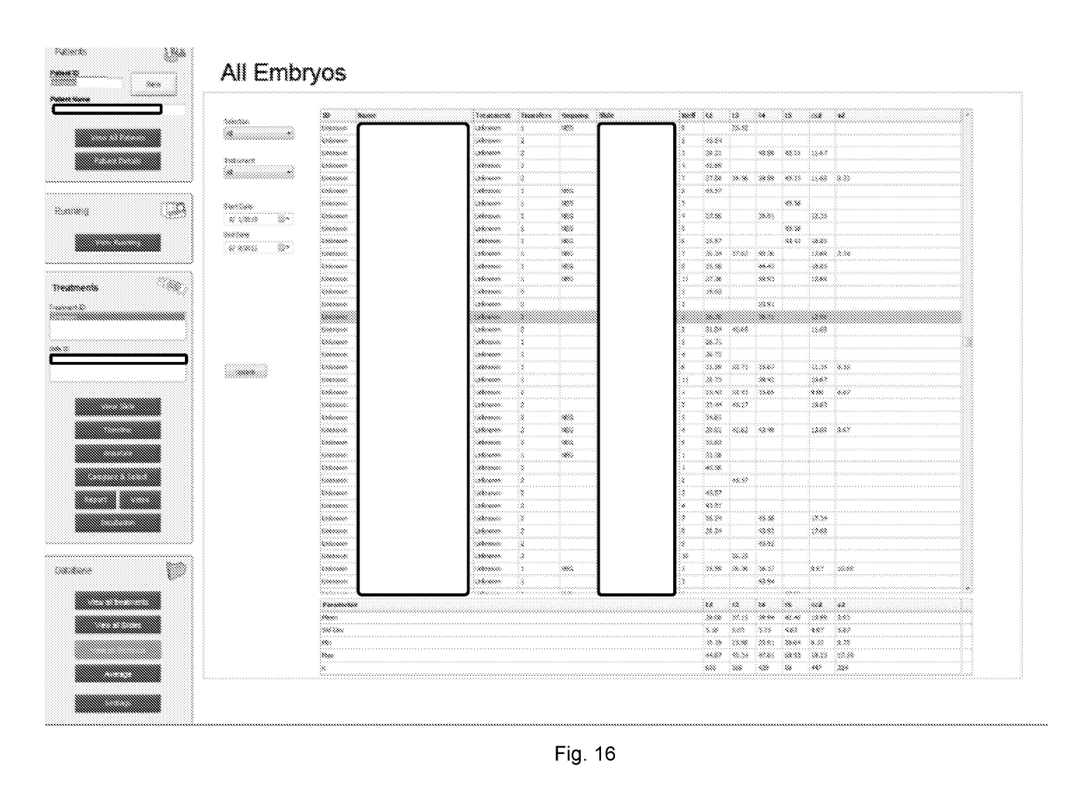

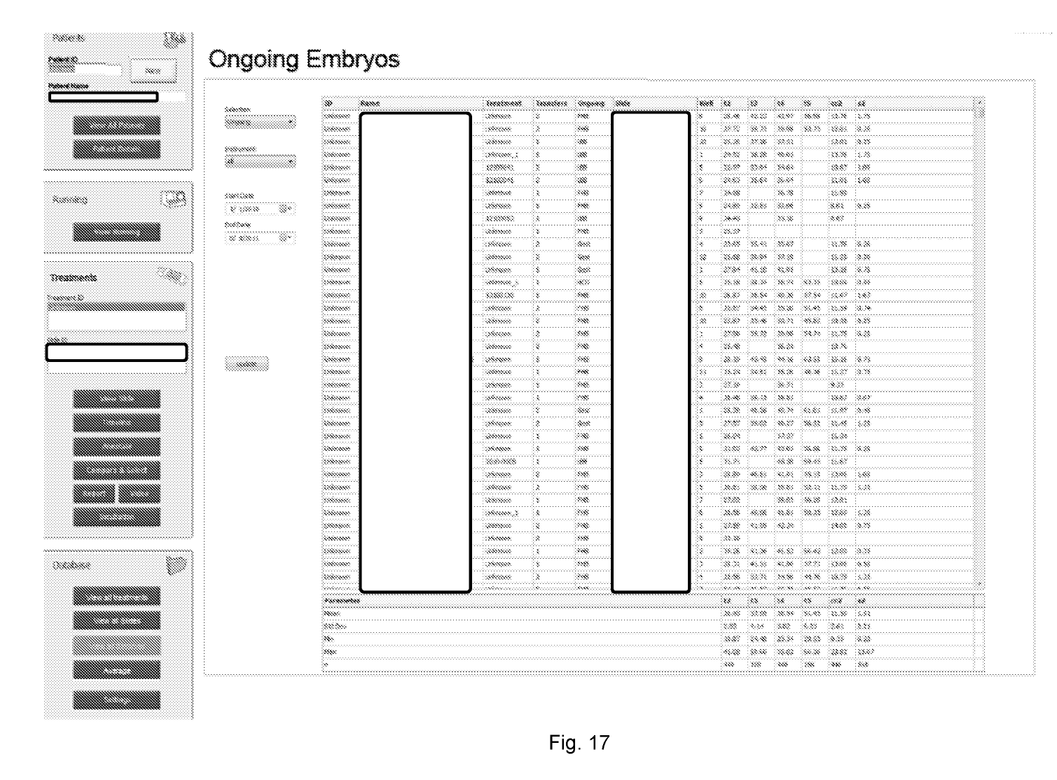

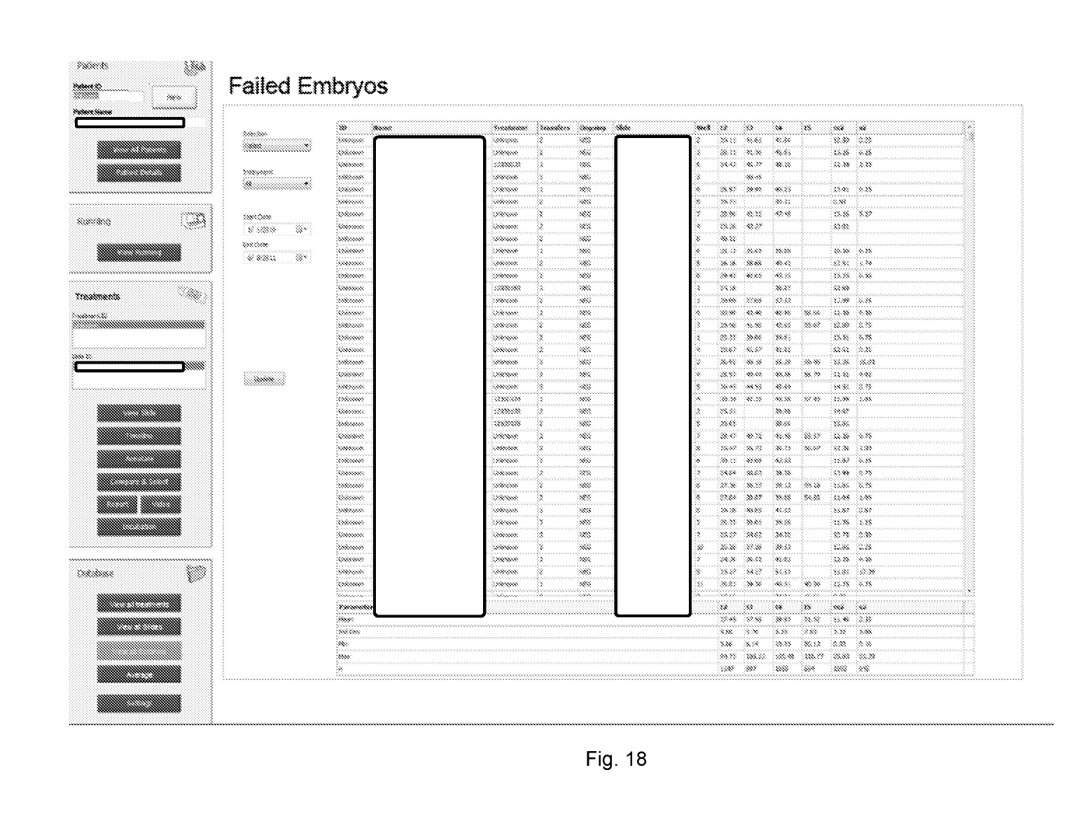

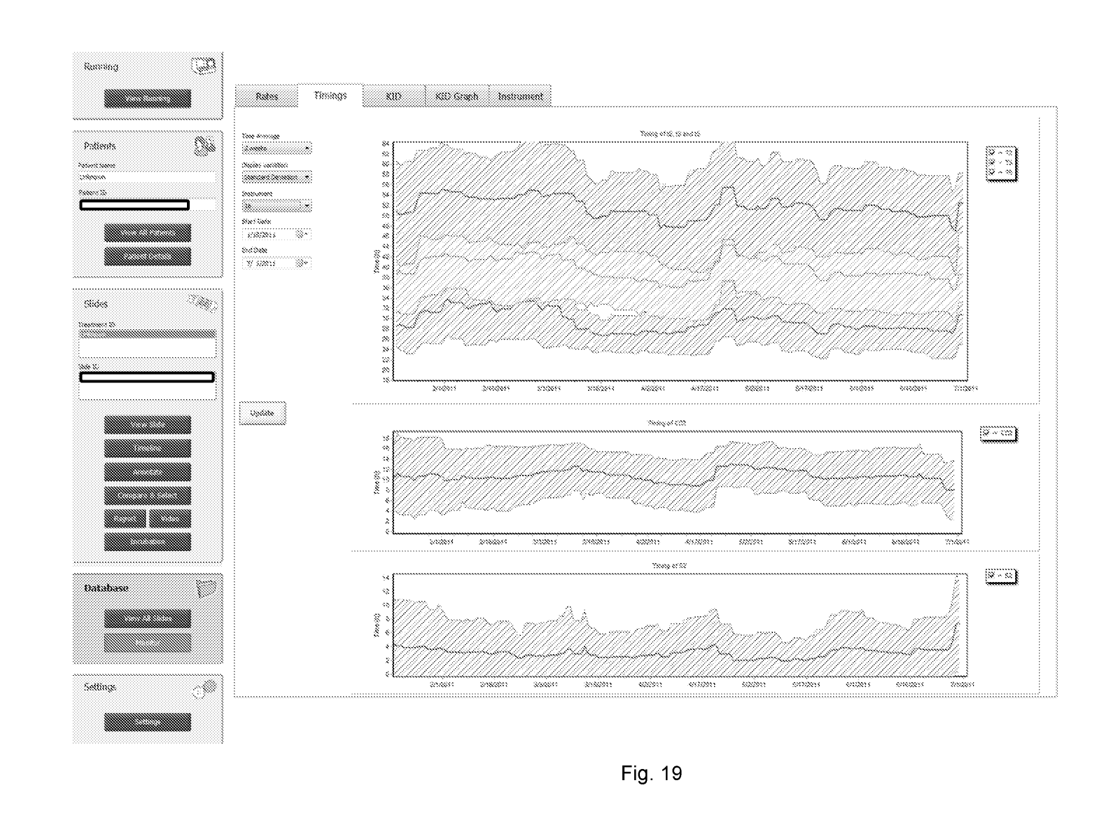

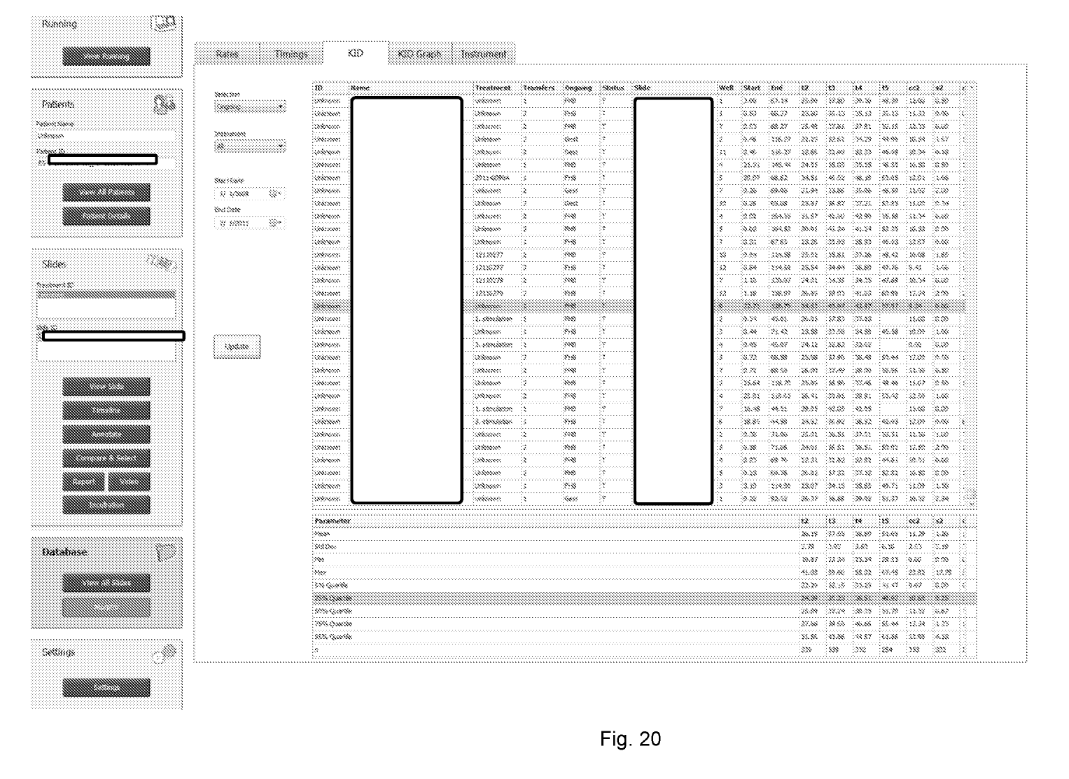

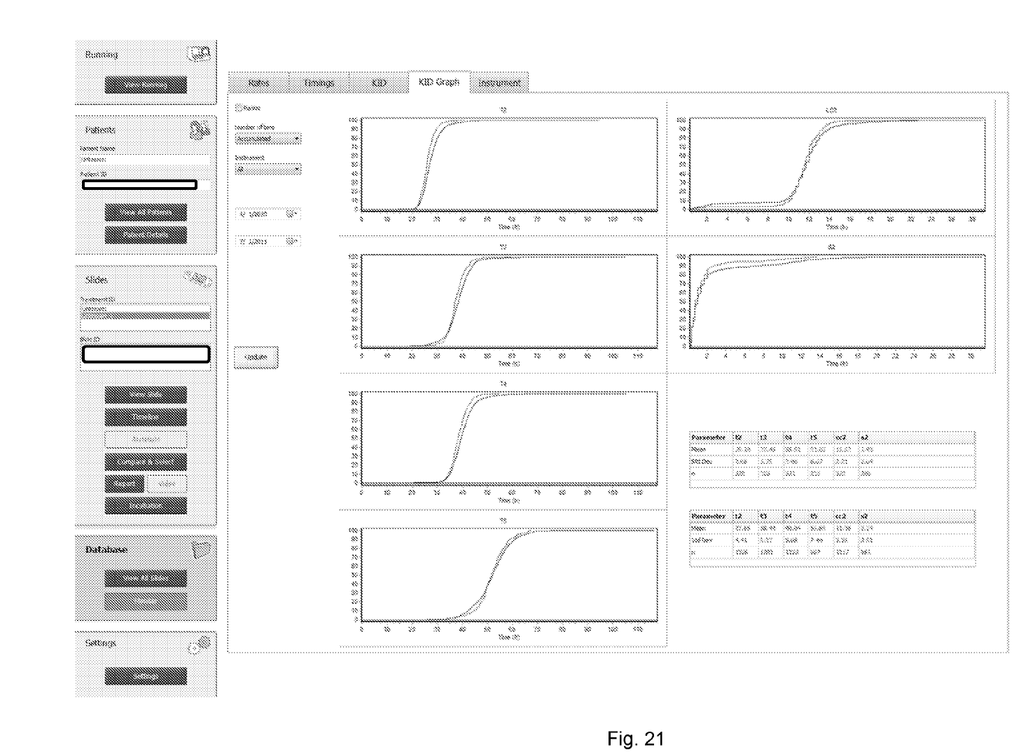

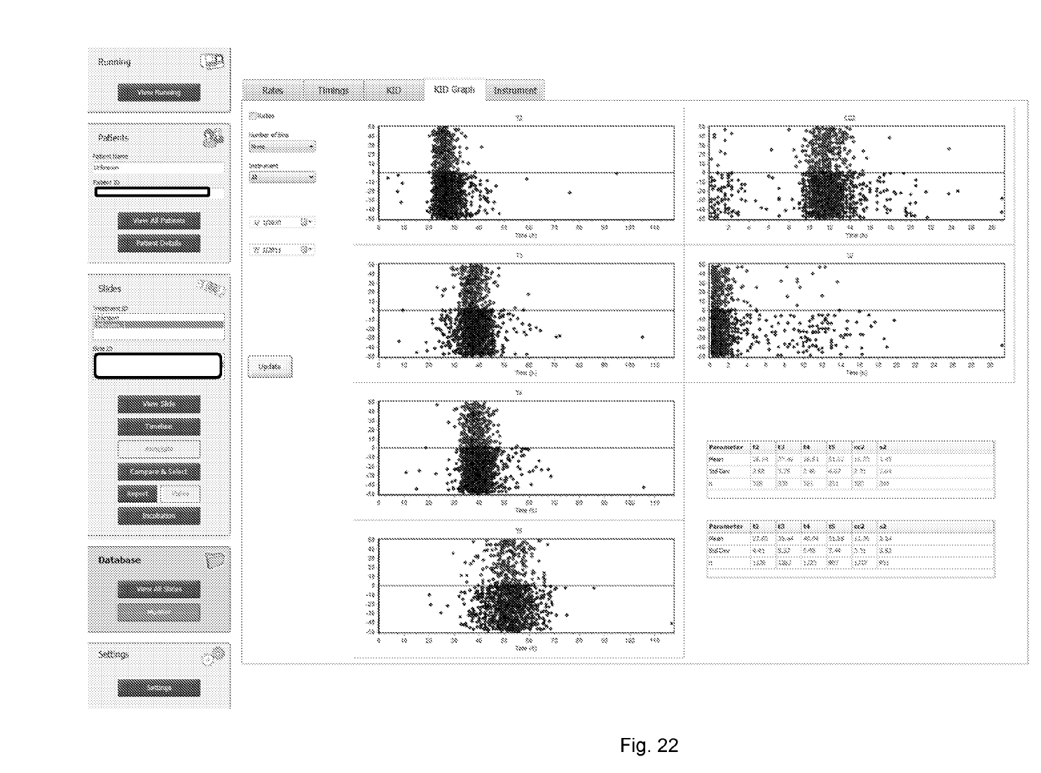

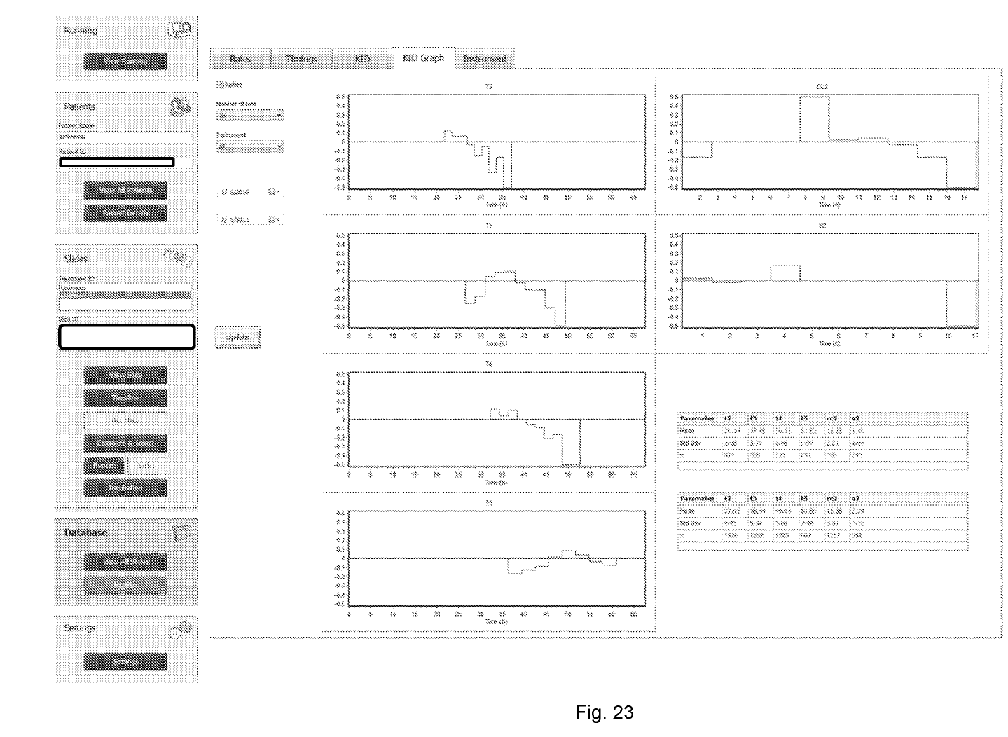

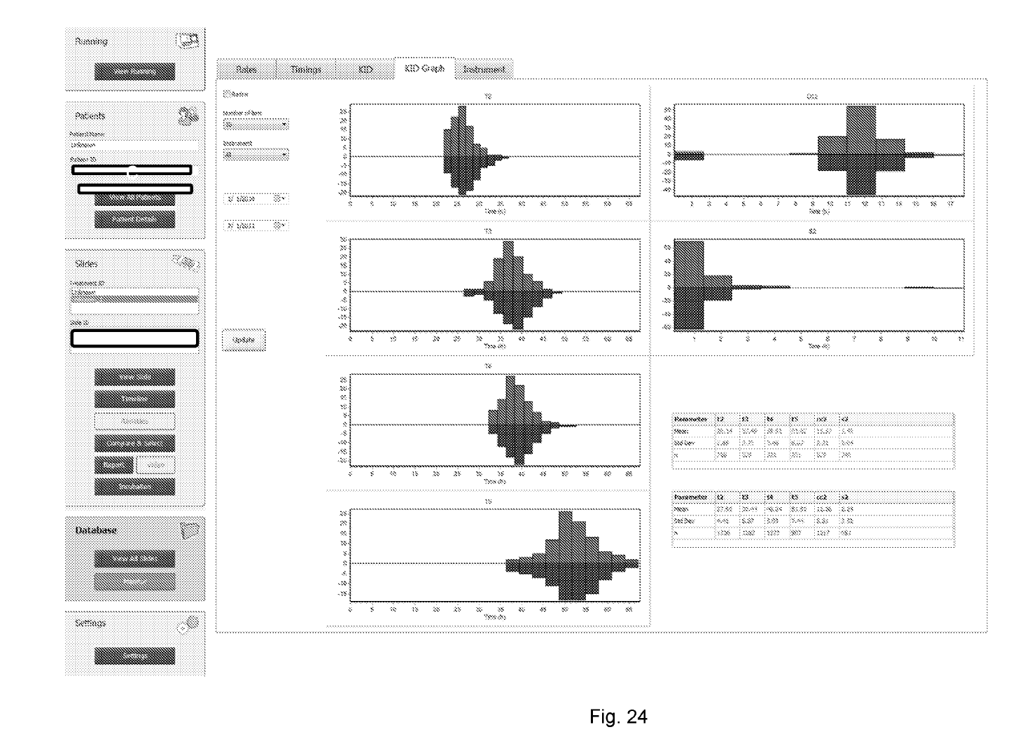

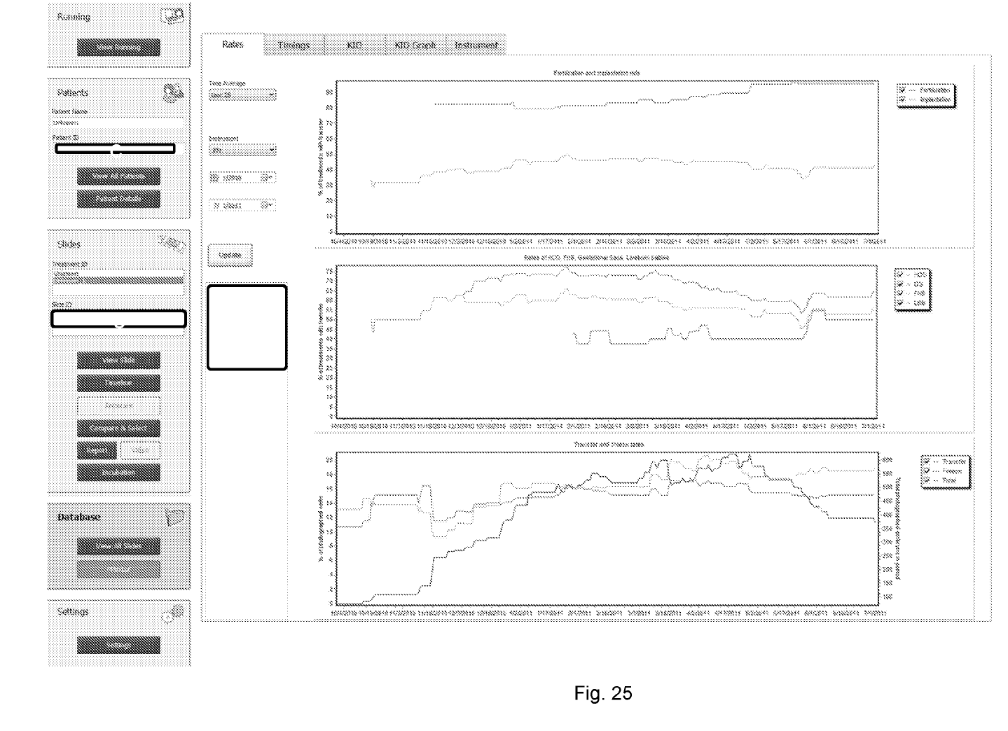

FIGS. 8a and 8b show the percentage of implanting embryos with cell division parameters below or above the median values. The two figures show classification for duration of second cell cycle (cc2) in FIG. 8a and synchrony of divisions from 2-cell to 4-cell stage (s2) in FIG. 8b. As the limits are defined as median values for all 247 investigated embryos with known implantation outcome, each column represent the same number of transferred embryos and the frequency of implantation was significantly higher for embryos with parameter values below the median.

### EXAMPLES

### Example 1

The principle of one embodiment of the invention is to adapt the quality criteria from the experienced clinic to the procedures used in the novice clinic by using morphokinetic information from all cleavage stage embryos in both clinics including those that were not transferred. A simple example would be to look at the timing of the first division from one to two cells, t2. Assuming:
1) The average division time for all cleavage stage embryos in the experienced clinic is: t2=27.5 hrs, and the standard deviation (StDev) is 1.5 hrs, based on cleavage time of 6000 developing embryos from 1000 treatments (as explained previously).
2) The average division time for all cleavage stage embryos in the novice clinic is: t2=26.5 hrs, and the standard deviation (StDev) is 1.0 hrs, based on the cleavage time of 300 embryos from 50 treatments.
3) The Experienced clinic has determined an optimal range for division to two cells for implanting embryos of 24.0 to 27.0 hrs. By comparing 1) and 2) the selection criteria for use in the novice clinic may be adapted as follows:
   a) The center of the selection range is transposed by the difference in average values between the clinics. The center of the interval from the experienced clinic was 25.5 hrs. The center for the novice clinic should consequently be 25.5+26.5−27.5=24.5 hrs.
   b) The range should be multiplied by the ratio of the StDev from the two clinics. Experienced clinic 27.0−24.0 hrs=3 hrs. The novel clinic would consequently be: 3.0 hrs*1.0hrs/1.5 hrs=2.0 hrs
   c) The adapted optimal range for the novice clinic would then become: 23.5 hrs to 25.5 hrs

Thus, the general procedure may e.g. comprise the following steps:
a) Identify a recognizable subpopulation of embryos from each clinic that constitute “Good Quality Embryos, GQE”. The criteria for GQE can be complex including multiple parameters (cell numbers at different timepoints, fragmentation, nucleation, etc.) or simple such as: more than six cells visible 68 hrs after insemination and fragmentation less than 20%. It is important that the same relevant group of likely viable embryos can be readily and unambiguously identified in both clinics.
b) Determine the morphokinetic parameters used in the selection criteria for GQE in both clinics.
c) Adapt the selection criteria from one clinic by accounting for the average difference in development of GQE between the two clinics. E.g. average estimates are modified by difference between average estimates of the two clinics. Ranges are modified by multiplication by the ratio of standard deviations between the clinics.
d) The criteria can be evaluated and if necessary by comparison with morphokinetic parameters from the (limited) number of embryos with known implantation from the novice clinic.

Different other scalings and assumptions can be envisioned, i.e. more rigorous transformations of distributions. The method can also be used to adapt selection methods published in the scientific literature to local protocol, provided the publication includes the relevant average and StDev measurements for recognizable GQE populations. It should be encouraged that future publications include this relevant information to the scientific and clinical community.

### Example 2

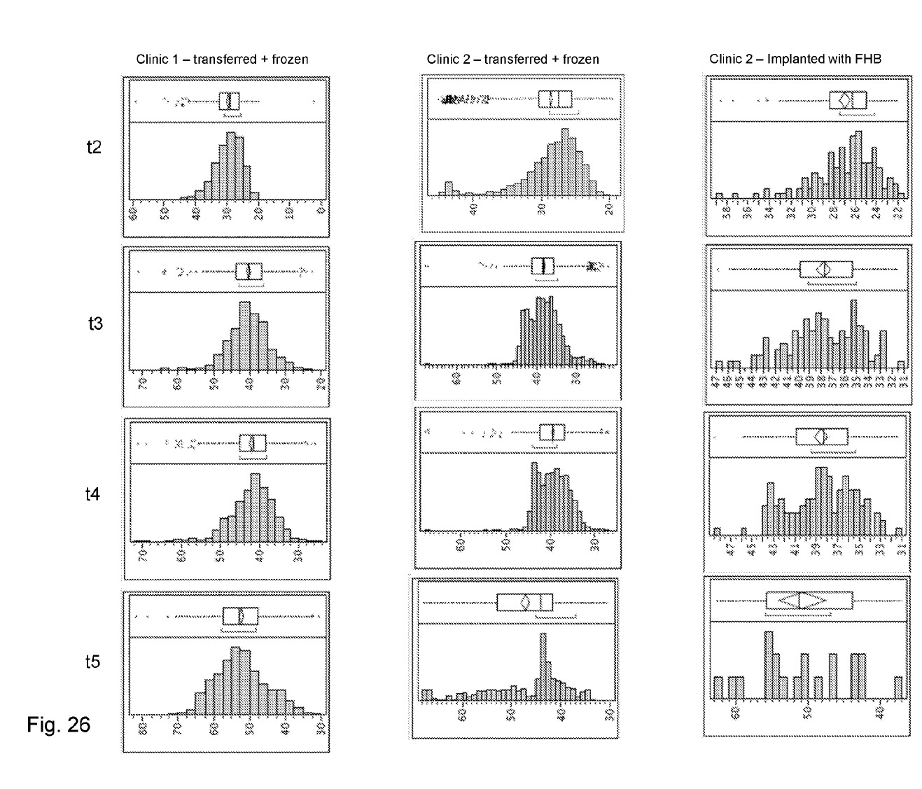

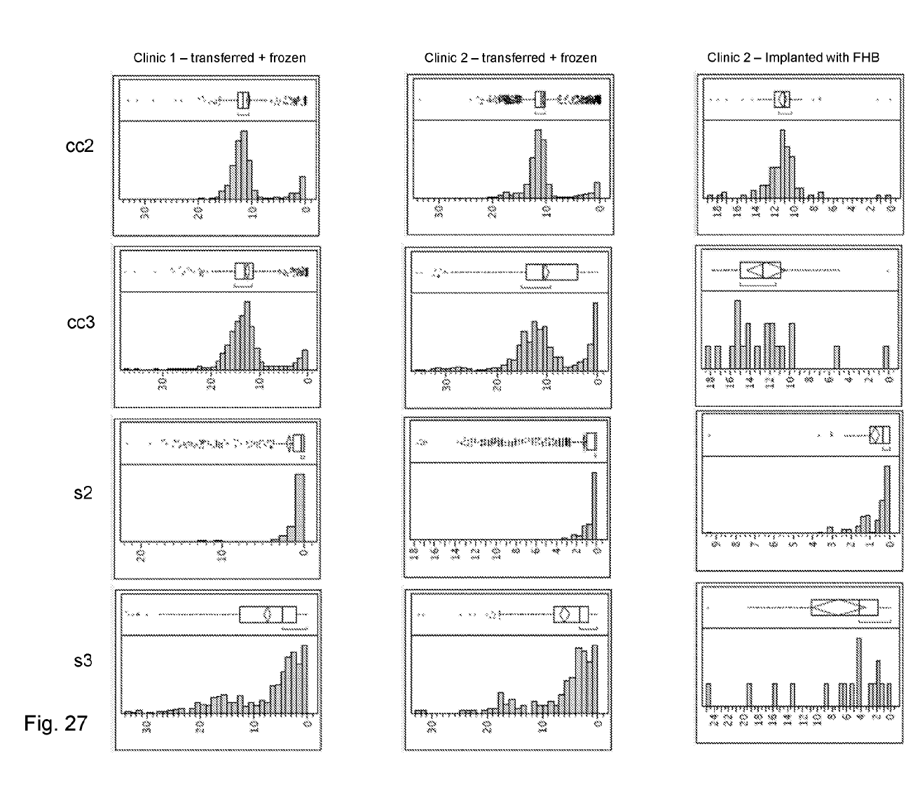

FIGS. 26 and 27 show statistical distributions for various cell division parameters where the data originate from two different fertility clinics; Clinic 1 and Clinic 2. Below are shown tables of statistical parameters calculated for various quality criteria with data originating from the two fertility clinics. Column “Clinic 1 T+F” is based on data from all transferred and frozen embryos from clinic 1, “Clinic 2 T+F” is based on data from all transferred and frozen embryos from clinic 2, and “Clinic 2 FHB” is based on data from successfully implanted embryos from clinic 2 where a fetal heart beat (FHB) has been registered. It is seen that the data basis for Clinic 2 is three to four times greater than the data basis for Clinic 1. By means of the present invention quality criteria has been calculated for Clinic 1. These are shown in the column “Clinic 1 Proposed” with the transposed center of the selection range and the adapted optimal range for the different quality criteria. In this example the quality criteria are the timing of cell divisions (t2, t3, t4 and t5), cell cycle durations (cc2 and cc3) and synchrony of cell divisions (s2 and s3). The statistical parameters are mean, standard deviation (Std Dev), standard error of the mean (Std Err Mean), 25, 50 and 75% quartile values and the total number of embryos (N). It is seen that N decreases when the embryo development progresses. That is because some of the embryos are selected for transfer earlier in their development.

| | t2 | | | | t3 | | | |
|---|---|---|---|---|---|---|---|---|
| Parameter | Clinic 1 | Clinic 2 | Clinic 2 | Clinic 1 | Clinic 1 | Clinic 2 | Clinic 2 | Clinic 1 |
| [hours] | T + F | T + F | FHB | Proposed | T + F | T + F | FHB | Proposed |
| Mean | 29.7 | 28.6 | 27.0 | 28.1 | 40.3 | 38.2 | 37.8 | 40.0 |
| Range | | | 23.9-30.0 | 25.0-31.2 | | | 34.7-41.0 | 35.5-44.4 |
| Std Dev | 4.8 | 4.7 | 3.1 | | 5.9 | 4.2 | 3.1 | |
| Std Err Mean | 0.2 | 0.1 | 0.3 | | 0.2 | 0.1 | 0.3 | |
| 75.0% quartile | 32.4 | 30.4 | 28.5 | | 43.8 | 41.3 | 39.8 | |
| 50.0% median | 29.1 | 27.5 | 26.4 | | 40.3 | 38.4 | 37.8 | |
| 25.0% quartile | 26.5 | 25.5 | 24.9 | | 36.6 | 35.7 | 35.3 | |
| N | 723 | 2656 | 124 | | 712 | 2317 | 117 | |

| | t4 | | | | t5 | | | |
|---|---|---|---|---|---|---|---|---|
| Parameter | Clinic 1 | Clinic 2 | Clinic 2 | Clinic 1 | Clinic 1 | Clinic 2 | Clinic 2 | Clinic 1 |
| [hours] | T + F | T + F | FHB | Proposed | T + F | T + F | FHB | Proposed |
| Mean | 42.0 | 39.3 | 38.5 | 41.2 | 47.2 | 52.3 | 50.8 | 45.6 |
| Range | | | 35.3-41.8 | 36.1-46.3 | | | 43.8-57.7 | 37.7-53.5 |
| Std Dev | 5.8 | 3.7 | 3.2 | | 8.5 | 7.5 | 7.0 | |
| Std Err Mean | 0.2 | 0.1 | 0.3 | | 0.4 | 0.3 | 1.6 | |
| 75.0% quartile | 45.1 | 42.1 | 40.8 | | 52.7 | 57.3 | 55.7 | |
| 50.0% median | 41.4 | 39.4 | 38.3 | | 43.9 | 52.8 | 51.2 | |
| 25.0% quartile | 38.5 | 36.7 | 36.1 | | 41.5 | 47.7 | 43.9 | |
| N | 703 | 2152 | 115 | | 476 | 631 | 20 | |

| | cc2 | | | | cc3 | | | |
|---|---|---|---|---|---|---|---|---|
| Parameter | Clinic 1 | Clinic 2 | Clinic 2 | Clinic 1 | Clinic 1 | Clinic 2 | Clinic 2 | Clinic 1 |
| [hours] | T + F | T + F | FHB | Proposed | T + F | T + F | FHB | Proposed |
| Mean | 10.7 | 12.0 | 11.2 | 9.9 | 12.5 | 10.4 | 12.4 | 14.4 |
| Range | | | 9.0-13.4 | 9.0-10.7 | | | 8.2-16.5 | 11.4-17.5 |
| Std Dev | 4.3 | 11.0 | 2.2 | | 5.1 | 7.1 | 4.2 | |
| Std Err Mean | 0.2 | 10.3 | 0.2 | | 0.2 | 0.3 | 0.9 | |
| 75.0% quartile | 12.7 | 12.0 | 12.0 | | 15.0 | 14.2 | 15.0 | |
| 50.0% median | 11.7 | 11.0 | 11.0 | | 13.0 | 11.0 | 12.7 | |
| 25.0% quartile | 10.7 | 10.3 | 10.5 | | 11.3 | 4.1 | 10.9 | |
| N | 712 | 2317 | 117 | | 631 | 476 | 20 | |

| | s2 | | | | s3 | | | |
|---|---|---|---|---|---|---|---|---|
| Parameter | Clinic 1 | Clinic 2 | Clinic 2 | Clinic 1 | Clinic 1 | Clinic 2 | Clinic 2 | Clinic 1 |
| [hours] | T + F | T + F | FHB | Proposed | T + F | T + F | FHB | Proposed |
| Mean | 1.8 | 1.3 | 0.8 | 1.3 | 7.5 | 6.0 | 7.1 | 8.6 |
| Range | | | 0-1.9 | 0-2.9 | | | 0.1-14.4 | 0.6-16.7 |
| Std Dev | 3.7 | 2.6 | 1.2 | | 7.3 | 6.4 | 7.1 | |
| Std Err Mean | 0.1 | 0.1 | 0.1 | | 0.3 | 0.5 | 1.7 | |
| 75.0% quartile | 1.3 | 1.0 | 1.0 | | 12.7 | 8.0 | 10.9 | |
| 50.0% median | 0.3 | 0.3 | 0.3 | | 4.7 | 3.3 | 4.3 | |
| 25.0% quartile | 0.0 | 0.0 | 0.0 | | 2.0 | 1.7 | 1.8 | |
| N | 703 | 2152 | 115 | | 548 | 196 | 17 | |

### Example 3

Development for three different groups of mouse embryos incubated in three different temperatures of the incubation medium were investigated under similar conditions, i.e. only the temperature differed between the three different groups. The temperature of the incubation media was assessed by measuring the temperature of the slideholder using a YSI precision thermometer.

The three different temperatures were 36.5° C. (33 embryos), 37.5° C. (63 embryos) and 38.5° C. (35 embryos), respectively. Nearly all mouse embryos reached the blastocyst stage as seen in the below table.

| Temperature of | | Blastocyst |
|---|---|---|
| slide holder (° C.) | N | rate (%) |
| 36.5 | 33 | 100 |
| 37.5 | 63 | 98 |
| 38.5 | 35 | 100 |

The table below shows the measured average timing for different cell divisions, the morula and blastocyst stage.

| Temp. | 2 cells | 3 cells | 4 cells | 5 cells | 6 cells | 7 cells | 8 cells | 9+ cells | | |
|---|---|---|---|---|---|---|---|---|---|---|
| (° C.) | (t2) | (t3) | (t4) | (t5) | (t6) | (t7) | (t8) | (t9) | Morula | Blastocyst |
| 36.5 | 4.61 | 26.36 | 27.57 | 35.91 | 36.30 | 37.10 | 37.54 | 44.54 | 49.45 | 67.46 |
| 37.5 | 3.75 | 23.43 | 24.25 | 32.12 | 32.54 | 33.19 | 33.63 | 40.72 | 45.85 | 59.27 |
| 38.5 | 3.06 | 21.96 | 22.50 | 30.62 | 30.97 | 31.43 | 31.87 | 39.06 | 44.29 | 55.03 |

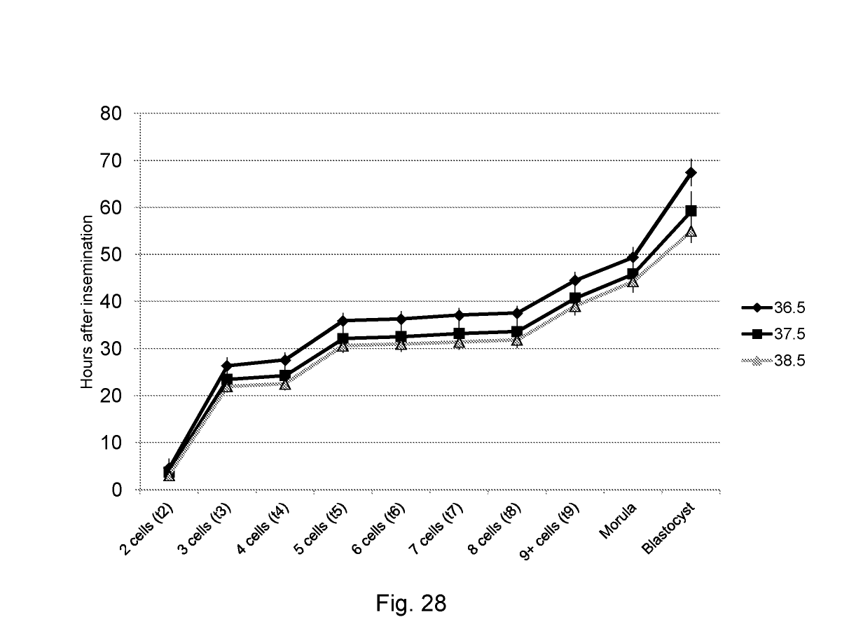

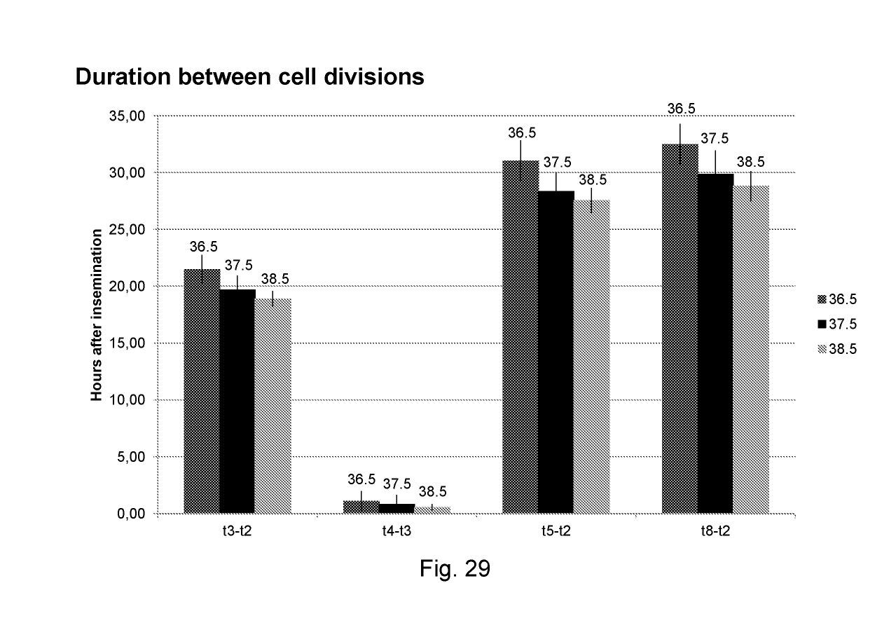

These data have been plotted in three graphs shown in FIG. 28. The difference between various cell divisions is shown in FIG. 29. The data and the graphs show that increasing the temperature of the medium clearly speeds up the development.

In order to assess the difference in development a relative rate coefficient k can be defined. If k is set to 1 at base temperature (T_{b}) the following relationship can be assumed:

k(T)=1+α*(T_{b}−36.5)

where Tis the temperature in ° C. and α is the temperature dependency coefficient.

The expected time t for a given temperature T, relative to t(T_{b}), is inversely proportional to k(T):

t(T)=t(T_{b})/k(T)

The above linear simplification offers the advantage of only requiring the estimation of a single parameter. Conversely, it is probably only valid within a narrow temperature range. However, in the case of human embryo incubation, the expected maximum temperature span would be somewhat below ±1° C., such that the practical influence of non-linearity can be considered negligible.

Optimising k(T) and t(T) by utilisation of the above listed mouse embryo data, using the time of division to 5 cells (t5), α is estimated to 0.080±0.015 (95% CI).

The Q₁₀ value is calculated as:

$${Q}_{10} = {( \frac{{R}_{2}}{{R}_{1}} )}^{10 / ( {T}_{2} - {T}_{1} )}$$

where R is the rate and T is the temperature.

Utilising the above parameter, the mouse embryo data, and the ±1° C. span in the experiment, the above equation yields a Q₁₀ of 2.22, which is inside the normally expected range of 2-3 in biological systems (Reyes et al., 2008, Mammalian peripheral circadian oscillators are temperature compensated. J. Biol. Rhythms 23: 95-98).

The same calculations have been performed for a set of data from 1397 human embryos extracted from different clinics. The incubation conditions for these human embryos are therefore not as similar as the above mentioned mouse embryos. However, the clinics belong to the same chain of IVF clinics using the same instrumentation. All embryos have been transferred with homogenised procedures, besides temperature. Utilising t5 here again, and optimising according to k(T) and t(T), the estimate for a becomes 0.058 ±0.028 (95% CI).

In contrast to the mouse embryos these human embryos have been incubated under slightly different conditions. The extracted human embryo data are therefore not comparable to the same degree as the mouse embryo data. However, again the data from the human embryos indicate that a higher temperature of the medium speeds up the development. This also shows the necessity for adapting embryo selection criteria to specific incubation conditions.

1. A method for determining one or more quality criteria for embryos being cultured under a first set of conditions, the method comprising the steps of:
a. providing
i. a first embryo dataset for embryos that have been cultured and/or monitored under said first set of conditions, and
ii. at least one second embryo dataset for embryos that have been cultured and/or monitored under at least a second set of conditions,
b. determining
i. a first group of statistical parameters by analysing said first embryo dataset,
ii. a second group of statistical parameters corresponding to the first group of statistical parameters by analysing said at least one second embryo dataset,
iii. one or more embryo quality criteria by analysing at least a subset of said at least one second embryo dataset; and
c. comparing the first group of statistical parameters to the second group of statistical parameters thereby detecting differences between the first and second group of statistical parameters: and
d. adapting said one or more embryo quality criteria derived from the second embryo dataset to be applicable for the first set of conditions based on differences detected between the first and second group of statistical parameters.

2. The method according to claim 1, further comprising the step of determining differences in conditions between the first set of conditions and the second set of conditions based on the detected differences between the first and second group of statistical parameters.

3-4. (canceled)

5. The method according to claim 1, wherein step b) further comprises the step of determining one or more embryo quality criteria by analysing a subset of said first embryo dataset.

6. The method according to claim 5, wherein the embryo quality criteria extracted from the first embryo dataset are the same type of embryo quality criteria extracted from the subset of the second embryo dataset.

7. The method according to claim 1, wherein said subset(s) of an embryo dataset comprise preimplantation data from implanted embryos that have resulted in ongoing pregnancies, live born babies, fetal heart beat (FHB), and/or gestational sacs.

8. The method according to claim 1, wherein the statistical parameters are selected from the group of mean, median, quartiles, standard deviation, ranges(min-max), percentiles and variance.

9. The method according to claim 1, wherein an embryo dataset comprise morphokinetic parameters for
1) all embryos in a group of monitored embryos, or
2) a functionally defined subgroup from the group of embryos.

10. The method according to claim 9, wherein the functionally defined subgroup of embryos are defined as:
all fertilized embryos in the group,
embryos that have divided to at least a predefined number of cells at a predefined number of hours after insemination, such as divided to at least 7 cells 68 hours after insemination,
embryos that have less than a predefined percentage of fragmentation at a predefined hours after insemination, e.g. less than 20% fragmentation 68 hours after insemination,
embryos that are not multinucleated at a predefined cell stage, e.g. at the four cell stage,
embryos that have been classified as “Good quality embryos” (GQE) by a qualified embryologist,
embryos that have been chosen for freeze or transfer,
embryos that have been chosen for transfer, and/or
embryos that have implanted.

11. The method according to claim 9, wherein the morphokinetic parameters are selected from the group of:
the timing and/or duration of cell-division periods and inter-division periods,—the timing and/or duration of: cleavage times, cleavage periods and cell cycle times;
the timing and/or duration of divisional stages and quiet stages,
synchrony of cell-divisions,
timing, extent or duration of cellular and/or organelle movement,
timing, extent or duration of late phase criteria.

12. The method according to claim, wherein said one or more embryo quality criteria extracted from the second embryo dataset is selected from the group of:
embryo quality criteria validated by additional datasets,
embryo quality criteria validated by retrospective studies,
embryo quality criteria validated by prospective studies,
embryo quality criteria validated by resampling,
embryo quality criteria validated by bootstrapping.

13-16. (canceled)

17. The method according to claim 1, wherein the first and second set of conditions correspond, respectively, to the conditions in two different devices for culturing and/or monitoring embryos.

18. The method according to claim 1, wherein the first and second embryo dataset originate, respectively, from two different devices for culturing and/or monitoring embryos.

19-20. (canceled)

21. The method according to claim 1, wherein said first embryos dataset is substantially smaller than the second embryo dataset, such as 2 times smaller, such as 5 times smaller, such as 10 times smaller, such as 50 times smaller, such as 100 times smaller, such as 200 times smaller, such as 500 times smaller, such as 1000 times smaller.

22. The method according to any of the preceding claim 1, wherein the embryos are monitored through image acquisition, such as image acquisition at least once per hour, such as image acquisition at least once per half hour, such as image acquisition at least once per twenty minutes, such as image acquisition at least once per fifteen minutes, such as image acquisition at least once per ten minutes, such as image acquisition at least once per five minutes, such as image acquisition at least once per two minutes, such as image acquisition at least once per minute.

23. The method according to any of the claim 1, wherein the embryos are monitored by means of time-lapse microscopy equipment.

24-25. (canceled)

26. A method for selecting an embryo suitable for transplantation, said method comprising obtaining embryo quality criteria according to any claim 1, and selecting the embryo having the highest embryo quality measure.

27. The method according to claim 26, further comprising the step of implanting the embryo.

28. A method for selecting one or more embryos suitable for freezing, said method comprising obtaining embryo quality criteria according to any of claim 1, and selecting the embryos having the highest embryo quality measures.

29. A system for determining embryo quality comprising means for carrying out the steps of claim 1.

30. A computer comprising computer code portions constituting means for executing a method according to claim 1.
